(19) United States
(12) Patent Application Publication
(10) Pub. No.: US 2021/0017160 A1
(43) Pub. Date: Jan. 21, 2021

(54) DEUTERATED sGC STIMULATORS

(71) Applicant: Cyclerion Therapeutics, Inc., Cambridge, MA (US)

(72) Inventors: Thomas Wai-Ho Lee, Lexington, MA (US); Song Xue, Newton, MA (US); Vishnu Vardhan Reddy Karnati, Sudbury, MA (US); Debra Jane Wallace, Lexington, MA (US)

(21) Appl. No.: 16/928,435

(22) Filed: Jul. 14, 2020

Related U.S. Application Data
(60) Provisional application No. 62/874,620, filed on Jul. 16, 2019.

(51) Int. Cl. C07D 403/04

(52) U.S. Cl. CPC C07D 403/04; C07B 2200/05

(57) ABSTRACT

This invention relates to deuterated compounds of Formula I, and pharmaceutically acceptable salts thereof.

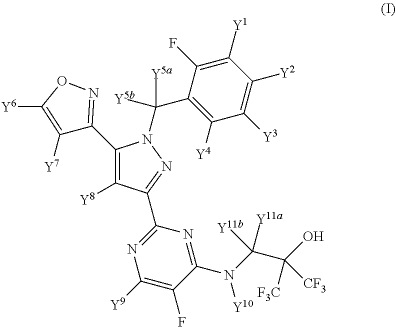

or a pharmaceutically acceptable salt thereof, wherein each of Y¹, Y², Y³, Y⁴, Y^{5a}, Y^{5b}, Y⁶, Y⁷, Y⁸, Y⁹, Y¹⁰, Y^{11a} and Y^{11b} is independently selected from hydrogen and deuterium, as well as pharmaceutical compositions, methods and uses thereof.

### RELATED APPLICATION

This application claims the benefit of the filing date, under 35 U.S.C. § 119(e), of U.S. Provisional Application No. 62/874,620, filed on Jul. 16, 2019. The entire contents of the above-referenced application are incorporated herein by reference.

### FIELD OF THE INVENTION

The present disclosure relates to stimulators of soluble guanylate cyclase (sGC), pharmaceutical formulations comprising them and their uses thereof, alone or in combination with one or more additional agents, for treating various diseases, wherein an increase in the concentration of nitric oxide (NO) or an increase in the concentration of cyclic guanosine 3′,5′-monophosphate (cGMP) or both, or an upregulation of the NO pathway is desirable.

### BACKGROUND OF THE INVENTION

sGC is the primary receptor for NO in vivo. Upon binding to sGC, NO activates its catalytic domain and results in the conversion of guanosine-5′-triphosphate (GTP) into the secondary messenger cGMP. The increased level of cGMP, in turn, modulates the activity of downstream effectors including protein kinases (e.g. PKG), phosphodiesterases (PDEs) and ion channels. In the body, NO is synthesized from L-arginine by various nitric oxide synthase (NOS) enzymes and by enzymatic reduction of inorganic nitrate. Experimental and clinical evidence indicates that reduced NO concentrations, reduced NO bioavailability and/or reduced responsiveness to endogenously produced NO contributes to the development of numerous diseases. sGC stimulators are a class of heme-dependent agonists of the sGC enzyme that work synergistically with varying amounts of NO to increase its enzymatic conversion of GTP to cGMP. sGC stimulators are clearly differentiated from and structurally unrelated to another class of NO-independent, heme-independent agonists of sGC known as sGC activators.

Therapies that improve or restore the function of sGC offer considerable advantages over current alternative therapies that either target the NO-sGC-PKG pathway or lead to the upregulation of the NO-sGC-PKG pathway. There is a need to develop novel stimulators of sGC. In particular, there is a need to develop stimulators of sGC with improved pharmacokinetic properties.

### SUMMARY OF THE INVENTION

In one aspect, the invention provides a compound represented by Formula I:

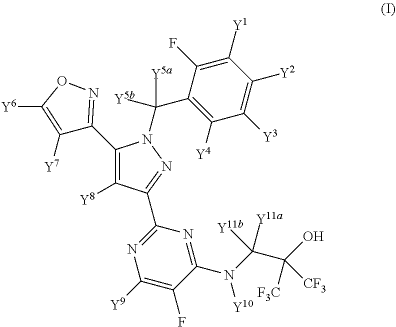

or a pharmaceutically acceptable salt thereof, wherein each of Y¹, Y², Y³, Y⁴, Y^{5a}, Y^{5b}, Y⁶, Y⁷, Y⁸, Y⁹, Y¹⁰, Y^{11a} and Y^{11b} is independently selected from hydrogen and deuterium; and at least one of Y¹, Y², Y³, Y⁴, Y^{5a}, Y^{5b}, Y⁶, Y⁷, Y⁸, Y⁹, Y¹⁰, Y^{11a} and Y^{11b} is deuterium.

In another embodiment, the invention relates to pharmaceutical compositions comprising a compound of Formula I, or a pharmaceutically acceptable salt thereof and at least one pharmaceutically acceptable excipient or carrier. In another embodiment, the invention relates to pharmaceutical dosage forms comprising said pharmaceutical compositions.

In another embodiment, the invention relates to a method of treating a disease in a subject in need thereof, comprising administering, alone or in combination therapy, a therapeutically effective amount of a compound of Formula I, or a pharmaceutically acceptable salt thereof, or pharmaceutical composition thereof, to the subject; wherein the disease is one that would benefit from sGC stimulation or from an increase in the concentration of NO and/or cGMP.

### DETAILED DESCRIPTION OF THE INVENTION

Compound 1,1,1,3,3,3-hexafluoro-2-(((5-fluoro-2-(1-(2-fluorobenzyl)-5-(isoxazol-3-yl)-1H-pyrazol-3-yl)pyrimidin-4-yl)amino)methyl)propan-2-ol, also known as praliciguat or IW-1973, is an orally administered sGC stimulator. Praliciguat is currently in Phase 2 clinical trials for the treatment of heart failure with preserved ejection fraction (HFpEF). It is also in a Phase 2 study in adult patients with diabetic nephropathy (DN).

Praliciguat has generally been well-tolerated in several clinical trials. Despite the potential beneficial activities of praliciguat, there is a continuing need for new compounds to treat the aforementioned diseases and conditions that have improved drug metabolism and pharmacokinetic (DMPK) properties. Improved DMPK properties have the potential to result in positive changes in safety profile, efficacy and tolerability of compounds.

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

### Definitions

For purposes of this disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry”, Thomas Sorrell, University Science Books, Sausalito: 1999, and “March's Advanced Organic Chemistry”, 5^{th} Ed., Smith, M. B. and March, J., eds. John Wiley & Sons, New York: 2001, which are herein incorporated by reference in their entirety.

It will be recognized that some variation of natural isotopic abundance occurs in a synthesized compound depending upon the origin of chemical materials used in the synthesis. Thus, a preparation of praliciguat will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66:15; and Gannes, L Z et al., Comp Biochem Physiol Mol Integr Physiol, 1998, 119:725.

The term “isotopic enrichment factor” at a particular position normally occupied by hydrogen refers to the ratio between the abundance of deuterium at the position and the natural abundance of deuterium at that position. By way of example, an isotopic enrichment factor of 3500 means that the amount of deuterium at the particular position is 3500-fold the natural abundance of deuterium, or that 52.5% of the compounds have deuterium at the particular position (i.e., 52.5% deuterium incorporation at the given position). The abundance of deuterium in the oceans of Earth is approximately one atom in 6500 hydrogen atoms (about 154 parts per million (ppm)). Deuterium thus accounts for approximately 0.015 percent (on a weight basis, 0.030 percent) of all naturally occurring hydrogen atoms in the oceans on Earth; the abundance changes slightly from one kind of natural water to another.

When a particular position in a compound of the invention (e.g., a compound represented by Formula I or a pharmaceutically acceptable salt thereof) is designated by name or structure as containing hydrogen or deuterium, it is to be understood that the position can contain hydrogen at its natural abundance or can be enriched in deuterium with an isotopic enrichment factor of, for example, at least 835 (12.5% deuterium incorporation), of at least 1670 (25% deuterium incorporation, of at least 3500 (52.5% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

When a particular position in a compound of the invention (e.g., a compound represented by Formula I or a pharmaceutically acceptable salt thereof) is designated specifically by name or structure as “H” or “hydrogen”, the position is understood to have hydrogen at its natural abundance isotopic composition.

When a particular position in a compound of the invention (e.g., a compound represented by Formula I or a pharmaceutically acceptable salt thereof) is designated specifically by name or structure as “D” or “deuterium”, the position is understood to have deuterium at an abundance that is at least 3340 times of the natural abundance of deuterium, which is 0.015% (i.e., at least 50.1% incorporation of deuterium), at least 3500 times of the natural abundance of deuterium (52.5% deuterium incorporation), at least 4500 times of the natural abundance of deuterium (67.5% deuterium incorporation), at least 5000 (75% deuterium), at least 5500 times of the natural abundance of deuterium (82.5% deuterium incorporation), at least 6000 times of the natural abundance of deuterium (90% deuterium incorporation), at least 6333.3 times of the natural abundance of deuterium (95% deuterium incorporation), at least 6466.7 times of the natural abundance of deuterium (97% deuterium incorporation), at least 6600 times of the natural abundance of deuterium (99% deuterium incorporation), or at least 6633.3 times of the natural abundance of deuterium (99.5% deuterium incorporation).

When a chemical name or structure is silent as to whether a particular position in a compound normally occupied by hydrogen is isotopically enriched, it is intended that the particular position is occupied by hydrogen at its natural abundance. By way of example, the term “phenyl” or

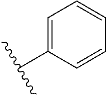 [SMILES: *c1ccccc1]

without any further designation as to isotopic enrichment indicates that all hydrogen atoms are present at natural abundance.

The term “compound,” when referring to any compound of this disclosure, including a compound represented by Formula I or a pharmaceutically acceptable salt thereof, refers to a collection of molecules having an identical chemical structure, except that there may be isotopic variation among the constituent hydrogen atoms of the molecules. The relative amount of isotopic variation in a compound of this invention will depend upon a number of factors including the isotopic purity of deuterated reagents used to make the compound and the efficiency of incorporation of deuterium in the various synthesis steps used to prepare the compound.

“D” and “d” both refer to deuterium. “H” refers to hydrogen.

“Substituted with deuterium” refers to the replacement of one or more hydrogen atoms with a corresponding number of deuterium atoms.

The invention also provides salts of the compounds of the invention (e.g., compound represented by Formula I). A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The compounds of the present invention (e.g., a compound represented by Formula I or a pharmaceutically acceptable salt thereof), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

### Compounds

Throughout this specification, a variable may be referred to generally (e.g., “each Y”) or may be referred to specifically (e.g., Y¹, Y², Y³, etc.). Unless otherwise indicated, when a variable is referred to generally, it is meant to include all specific embodiments of that particular variable.

In a first embodiment, the present disclosure is directed to a compound of Formula I:

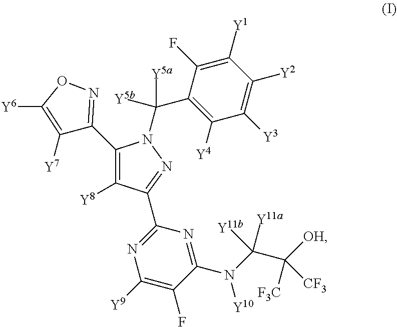

or a pharmaceutically acceptable salt thereof, wherein each of Y¹, Y², Y³, Y⁴, Y^{5a}, Y^{5b}, Y⁶, Y⁷, Y⁸, Y⁹, Y¹⁰, Y^{11a} and Y^{11b} is independently selected from hydrogen and deuterium; and at least one of Y¹, Y², Y³, Y⁴, Y^{5a}, Y^{5b}, Y⁶, Y⁷, Y⁸, Y⁹, Y¹⁰, Y^{11a} and Y^{11b} is deuterium.

In a second embodiment, in a compound of Formula I or a pharmaceutically acceptable salt thereof in accordance with the first embodiment, at least one of Y¹, Y², Y³ and Y⁴ is deuterium.

In a third embodiment, in a compound of Formula I or a pharmaceutically acceptable salt thereof in accordance with the first or second embodiment, at least one of Y^{5a} and Y^{5b} is deuterium.

In a fourth embodiment, in a compound of Formula I or a pharmaceutically acceptable salt thereof in accordance with the first, second or third embodiment, at least one of Y⁶ and Y⁷ is deuterium.

In a fifth embodiment, in a compound of Formula I or a pharmaceutically acceptable salt thereof in accordance with the first, second, third or fourth embodiment, at least one of Y^{11a} and Y^{11b} is deuterium.

In a sixth embodiment, in a compound of Formula I or a pharmaceutically acceptable salt thereof in accordance with the first, second, third, fourth or fifth embodiment, each of Y¹, Y², Y³ and Y⁴ are the same.

In a seventh embodiment, in a compound of Formula I or a pharmaceutically acceptable salt thereof in accordance with the first, second, third, fourth, fifth or sixth embodiment, each of Y^{5a} and Y^{5b} are the same.

In an eighth embodiment, in a compound of Formula I or a pharmaceutically acceptable salt thereof in accordance with the first, second, third, fourth, fifth, sixth or seventh embodiment, each of Y⁶ and Y⁷ are the same.

In a ninth embodiment, in a compound of Formula I or a pharmaceutically acceptable salt thereof in accordance with the first, second, third, fourth, fifth, sixth, seventh or eighth embodiment, each of Y^{11a} and Y^{11b} are the same.

In a tenth embodiment, in a compound of Formula I or a pharmaceutically acceptable salt thereof in accordance with the first, second, third, fourth, fifth, sixth, seventh, eighth, or ninth embodiment, Y¹ is deuterium.

In an eleventh embodiment, in a compound of Formula I or a pharmaceutically acceptable salt thereof in accordance with the first, second, third, fourth, fifth, sixth, seventh, eighth, ninth, or tenth embodiment, Y² is deuterium.

In a twelfth embodiment, in a compound of Formula I or a pharmaceutically acceptable salt thereof in accordance with the first, second, third, fourth, fifth, sixth, seventh, eighth, ninth, tenth, or eleventh embodiment, Y³ is deuterium.

In a thirteenth embodiment, in a compound of Formula I or a pharmaceutically acceptable salt thereof in accordance with the first, second, third, fourth, fifth, sixth, seventh, eighth, ninth, tenth, eleventh, or twelfth embodiment, Y⁴ is deuterium.

In a fourteenth embodiment, in a compound of Formula I or a pharmaceutically acceptable salt thereof in accordance with the first, second, third, fourth, fifth, sixth, seventh, eighth, ninth, tenth, eleventh, twelfth, or thirteenth embodiment, Y^{11a} is deuterium.

In a fifteenth embodiment, in a compound of Formula I or a pharmaceutically acceptable salt thereof in accordance with the first, second, third, fourth, fifth, sixth, seventh, eighth, ninth, tenth, eleventh, twelfth, thirteenth, or fourteenth embodiment, Y^{11b} is deuterium.

In a sixteenth embodiment, the compound of Formula I in accordance with the first embodiment or any other preceding embodiment, is selected from any one of the compounds (Cmpd) set forth in Table 1 (below) or a pharmaceutically acceptable salt thereof:

TABLE 1
Exemplary Embodiments of Formula I

| Cmpd | Y¹ | Y² | Y³ | Y⁴ | Y^{11a} | Y^{11b} |
|---|---|---|---|---|---|---|
| I-101 | D | H | H | H | H | H |
| I-102 | H | D | H | H | H | H |
| I-103 | H | H | D | H | H | H |
| I-104 | H | H | H | D | H | H |
| I-105 | H | H | H | H | D | H |
| I-106 | H | H | H | H | H | D |
| I-107 | D | D | H | H | H | H |
| I-108 | D | H | D | H | H | H |
| I-109 | D | H | H | D | H | H |
| I-110 | D | H | H | H | D | H |
| I-111 | D | H | H | H | H | D |
| I-112 | H | D | D | H | H | H |
| I-113 | H | D | H | D | H | H |
| I-114 | H | D | H | H | D | H |
| I-115 | H | D | H | H | H | D |
| I-116 | H | H | D | D | H | H |
| I-117 | H | H | D | H | D | H |
| I-118 | H | H | D | H | H | D |
| I-119 | H | H | H | D | D | H |
| I-120 | H | H | H | D | H | D |
| I-121 | H | H | H | H | D | D |
| I-122 | D | D | D | H | H | H |
| I-123 | D | D | H | D | H | H |
| I-124 | D | D | H | H | D | H |
| I-125 | D | D | H | H | H | D |
| I-126 | D | H | D | D | H | H |
| I-127 | D | H | D | H | D | H |
| I-128 | D | H | D | H | H | D |
| I-129 | D | H | H | D | D | H |
| I-130 | D | H | H | D | H | D |
| I-131 | D | H | H | H | D | D |
| I-132 | H | D | D | D | H | H |
| I-133 | H | D | D | H | D | H |
| I-134 | H | D | D | H | H | D |
| I-135 | H | D | H | D | D | H |
| I-136 | H | D | H | D | H | D |
| I-137 | H | D | H | H | D | D |
| I-138 | H | H | D | D | D | H |
| I-139 | H | H | D | D | H | D |
| I-140 | H | H | D | H | D | D |
| I-141 | H | H | H | D | D | D |
| I-142 | H | H | D | D | D | D |
| I-143 | H | D | H | D | D | D |
| I-144 | H | D | D | H | D | D |
| I-145 | H | D | D | D | H | D |
| I-146 | D | H | D | D | D | H |
| I-147 | D | H | H | D | D | D |
| I-148 | D | H | D | H | D | D |
| I-149 | D | H | D | D | H | D |
| I-150 | D | H | D | D | D | H |
| I-151 | D | D | H | H | D | D |
| I-152 | D | D | H | D | H | D |
| I-153 | D | D | H | D | D | H |
| I-154 | D | D | D | H | H | D |
| I-155 | D | D | D | H | D | H |
| I-156 | D | D | D | D | H | H |
| I-157 | D | D | D | D | D | H |
| I-158 | D | D | D | D | H | D |
| I-159 | D | D | D | H | D | D |
| I-160 | D | D | H | D | D | D |
| I-161 | H | D | D | D | D | D |
| I-162 | D | H | D | D | D | D |
| I-163 | D | D | H | D | D | D |
| I-164 | D | D | D | D | D | D |

In a specific embodiment, for compounds I-101 to I-164 described in Table 1 above, Y^{5a}, Y^{5b}, Y⁶, Y⁷, Y⁸, Y⁹, Y¹⁰, Y^{12a} and Y^{12b} are all H.

In a seventeenth embodiment, the compound of Formula I in accordance with the first or sixteenth embodiment or any other preceding embodiment, is selected from any one of the following compounds or a pharmaceutically acceptable salt thereof:

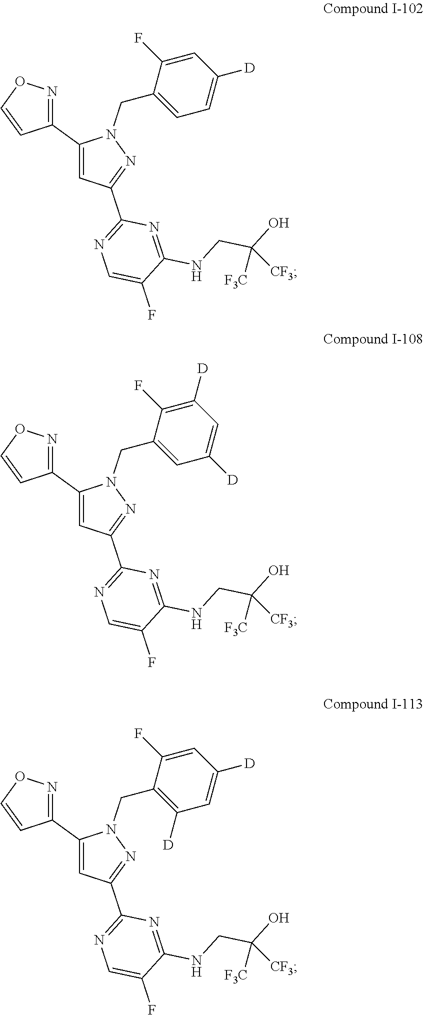 [SMILES: [2H]c1ccc(Cn2nc(-c3ncc(F)c(NCC(O)(C(F)(F)F)C(F)(F)F)n3)cc2-c2ccon2)c(F)c1 ; [2H]c1cc([2H])c(F)c(Cn2nc(-c3ncc(F)c(NCC(O)(C(F)(F)F)C(F)(F)F)n3)cc2-c2ccon2)c1 ; [2H]c1cc([2H])c(Cn2nc(-c3ncc(F)c(NCC(O)(C(F)(F)F)C(F)(F)F)n3)cc2-c2ccon2)c(F)c1]

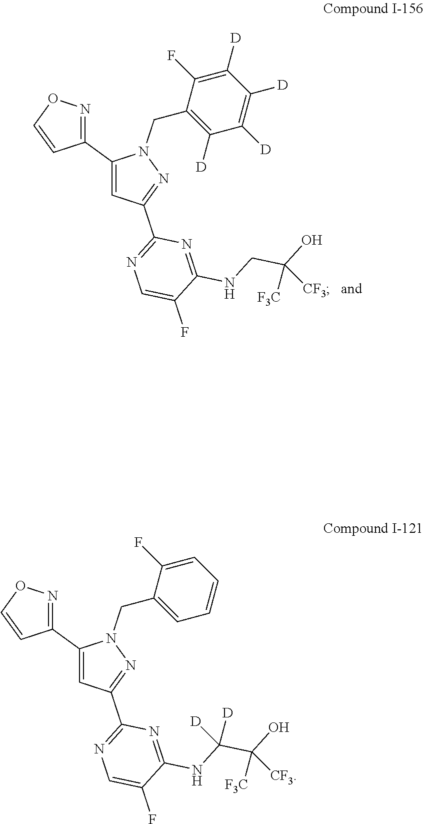 [SMILES: [2H]c1c([2H])c([2H])c(Cn2nc(-c3ncc(F)c(NCC(O)(C(F)(F)F)C(F)(F)F)n3)cc2-c2ccon2)c(F)c1[2H] ; "and" ; [2H]C([2H])(Nc1nc(-c2cc(-c3ccon3)n(Cc3ccccc3F)n2)ncc1F)C(O)(C(F)(F)F)C(F)(F)F]

In an eighteenth embodiment, in a compound of Formula I in accordance with the first or second embodiment or any other preceding embodiment, when any one of Y¹, Y², Y³ and Y⁴ is deuterium, the level of deuterium incorporation at each of Y¹, Y², Y³ and Y⁴ designated as deuterium is at least 52.5%, at least 75%, at least 82.5%, at least 90%, at least 95%, at least 97%, at least 98%, or at least 99%.

In a nineteenth embodiment, in a compound of Formula I in accordance with the first or third embodiment or any other preceding embodiment, when any one of Y^{5a} and Y^{5b} is deuterium, the level of deuterium incorporation at each of Y^{5a} and Y^{5b} is at least 52.5%, at least 75%, at least 82.5%, at least 90%, at least 95%, at least 97%, at least 98%, or at least 99%.

In a twentieth embodiment, in a compound of Formula I in accordance with the first or fourth embodiment or any other preceding embodiment, when any one of Y⁶ and Y⁷ is deuterium, the level of deuterium incorporation at each of Y⁶ and Y⁷ is at least 52.5%, at least 75%, at least 82.5%, at least 90%, at least 95%, at least 97%, at least 98%, or at least 99%.

In a twenty-first embodiment, in a compound of Formula I in accordance with the first or fifth embodiment or any other preceding embodiment, when any one of Y^{11a} and Y^{11b} is deuterium, the level of deuterium incorporation at each of Y^{11a} and Y^{11b} is at least 52.5%, at least 75%, at least 82.5%, at least 90%, at least 95%, at least 97%, at least 98%, or at least 99%.

In one embodiment, any atom not designated as deuterium in any of the preceding embodiments set forth above is present at its natural isotopic abundance.

In one embodiment, in a compound of the invention as set forth in any of the preceding embodiments, the level of deuterium incorporation at each designated deuterium position is at least 52.5%, at least 75%, at least 82.5%, at least 90%, at least 95%, at least 97%, at least 98%, or at least 99%.

In one embodiment, of a compound of this invention, at least one of Y¹, Y², Y³, Y⁴, Y^{5a} Y^{5b}, Y⁶, Y⁷, Y⁸, Y⁹, Y¹⁰, Y^{11a} and Y^{11b} is hydrogen.

The synthesis of compounds of Formula I may be readily achieved by synthetic chemists of ordinary skill by reference to the Exemplary Synthesis and Examples disclosed herein. Relevant procedures analogous to those of use for the preparation of compounds of Formula I and intermediates thereof are disclosed, for instance in International Patent Application Publication Nos. WO 2012/003405, WO 2013/101830, WO 2014/144100, WO 2018/009597, WO 2018/009602, and International Patent Application No. PCT/US2019/013060, each of which is incorporated herein by reference.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Synthesis

Exemplary methods for synthesizing a compound of Formula I are depicted in Scheme I:

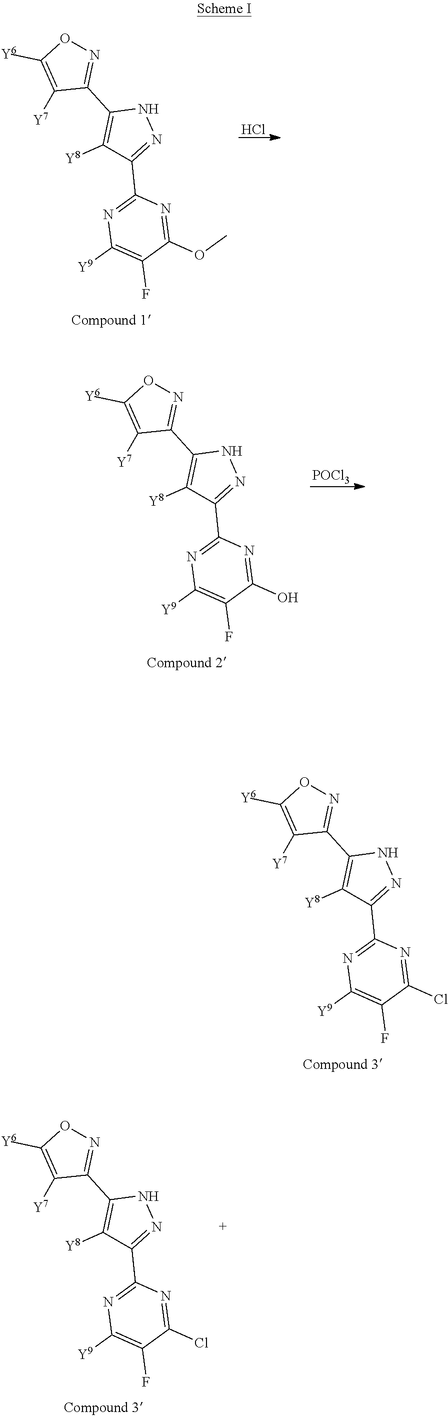

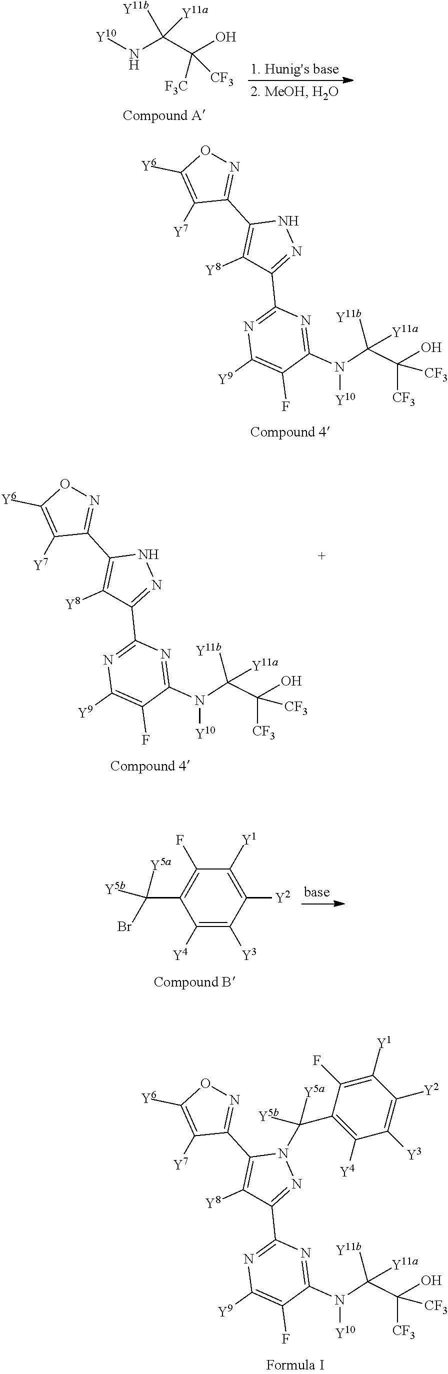

Each of Y¹, Y², Y³, Y⁴, Y^{5a}, Y^{5b}, Y⁶, Y⁷, Y⁸, Y⁹, Y¹⁰, Y^{11a} and Y^{11b} in Compounds 1′, 2′, 3′, 4′, A′, B′ and Formula I, as depicted in Scheme I, is independently selected from hydrogen and deuterium. Briefly, Compound 1′ is demethylated in the presence of concentrated hydrochloric acid to yield Compound 2′. Compound 2′ is then chlorinated by reacting with a chlorination reagent (e.g. POCl₃) to produce Compound 3′. Next, Compound 3′ is coupled with amine Compound A′ in the presence of a base (e.g., Hunig's base or N,N-diisopropylethylamine DIPEA or DIEA) to yield Compound 4′. Finally, Compound 4′ is reacted with Compound B′ or a similar alkylating agent in the presence of a base (e.g., lithium tert-butoxide, sodium tert-butoxide or potassium tert-butoxide), to yield a compound of Formula I.

### Pharmaceutically Acceptable Salts

“Pharmaceutically acceptable salts” of the compounds described herein include those derived from said compounds when mixed with inorganic or organic acids or bases. In some embodiments, the salts can be prepared in situ during the final isolation and purification of the compounds. In other embodiments the salts can be prepared from the free form of the compound in a separate synthetic step. The preparation of the pharmaceutically acceptable salts described above, and other typical pharmaceutically acceptable salts is more fully described by Berg et al., “Pharmaceutical Salts,” J. Pharm. Sci., 1977:66:1-19, incorporated here by reference in its entirety. The pharmaceutically acceptable salts of a compound of Formula I are those that may be used in medicine. Salts that are not pharmaceutically acceptable may, however, be useful in the preparation of compounds of Formula I or of their pharmaceutically acceptable salts.

When a compound of Formula I is acidic, suitable “pharmaceutically acceptable salts” refers to salts prepared from pharmaceutically acceptable non-toxic bases including inorganic and organic bases. Salts derived from inorganic bases include aluminum, ammonium, calcium, copper, ferric, ferrous, lithium, magnesium, manganic, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines, arginine, betaine, caffeine, choline, N, N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When a compound of Formula I is basic, salts may be prepared from pharmaceutically acceptable non-toxic acids, including inorganic and organic acids. Such acids include acetate, acetic, acid citrate, acid phosphate, ascorbate, benzenesulfonic, benzenesulfonate, benzoic, benzoate, bromide, bisulfate, bitartrate, camphorsulfonic, chloride, citrate, citric, ethanesulfonate, ethanesulfonic, formate, fumarate, fumaric, gentisinate, gluconate, gluconic, glucuronate, glutamate, glutamic, hydrobromic, hydrochloric, iodide, isethionic, isonicotinate, lactate, lactic, maleate, maleic, malic, mandelic, methanesulfonic, methanesulfonate, mucic, nitrate, nitric, oleate, oxalate, pamoic, pamoate (i.e., 1,1′-methylene-bis-(2-hydroxy-3-naphthoate)), pantothenic, pantothenate, phosphate, phosphoric, saccharate, salicylate, succinic, succinate, sulfuric, sulfate, tannate, tartrate, tartaric, p-toluenesulfonate, p-toluenesulfonic acid and the like. Particular embodiments include citric, hydrobromic, hydrochloric, maleic, phosphoric, sulfuric and tartaric acids.

In addition to the compounds described herein, their pharmaceutically acceptable salts may also be employed in compositions to treat or prevent the herein identified diseases.

### Pharmaceutical Compositions and Methods of Administration

The compounds herein disclosed, and their pharmaceutically acceptable salts thereof may be formulated as pharmaceutical compositions or “formulations”.

A typical formulation is prepared by mixing a compound of Formula I or a pharmaceutically acceptable salt thereof and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which a compound of Formula I is being formulated. Solvents are generally selected based on solvents recognized by persons skilled in the art as safe (GRAS-Generally Regarded as Safe) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g. enteric or slow release) preservatives, antioxidants, opaquing agents, glidants, processing aids, colorants, sweeteners, perfuming agents, flavoring agents and other known additives to provide an elegant presentation of the drug (i.e., a compound of Formula I or pharmaceutical composition thereof) or aid in the manufacturing of the pharmaceutical product (i.e., medicament).

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively; in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

The formulations may be prepared using conventional dissolution and mixing procedures. The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations, and is the minimum amount necessary to ameliorate, cure or treat the disease, or one or more of its symptoms.

The terms “administer”, “administering” or “administration” in reference to a compound, composition or dosage form of the invention means introducing the compound into the system of the subject or patient in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g. orally (including, but not limited to solid dosage forms including hard or soft capsules (e.g. gelatin capsules), tablets, pills, powders, sublingual tablets, troches, lozenges, and granules; and liquid dosage forms including, but are not limited to, pharmaceutically acceptable emulsions, microemulsions, aqueous or oil solutions, suspensions, syrups and elixirs, by inhalation (e.g. with an aerosol, gas, inhaler, nebulizer or the like), to the ear (e.g. using ear drops), topically (e.g. using creams, gels, inhalants, liniments, lotions, ointments, patches, pastes, powders, solutions, sprays, transdermal patches, etc.), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc.), via ear drops, via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Formulations of a compound intended for oral use may be prepared according to any method known to the art for the manufacture of pharmaceutical compositions.

In solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

The oral compositions (either solid or liquid) can also include excipients and adjuvants such as dispersing or wetting agents, such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate); emulsifying and suspending agents, such as sodium carboxymethylcellulose, croscarmellose, povidone, methylcellulose, hydroxypropyl methylcelluose, sodium alginate, polyvinylpyrrolidone, gum tragacanth and gum acacia; sweetening, flavoring, and perfuming agents; and/or one or more preservatives such as ethyl or n-propyl p-hydroxy-benzoate, one or more coloring agents, one or more flavoring agents and one or more sweetening agents, such as sucrose or saccharin.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration have a particle size for example in the range of 0.1 to 500 micros (including particles in a range between 0.1 and 500 microns in increments microns such as 0.5, 1, 30, 35 microns, etc.) which is administered by rapid inhalation through the nasal passage or by inhalation through the mouth so as to reach the alveolar sacs.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs. The active component is admixed under sterile conditions with a pharmaceutically acceptable carrier and any needed preservatives or buffers as may be required.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using a compound of Formula I may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of a compound of Formula I include Tween™-60, Span™-80, cetostearyl alcohol, benzyl alcohol, myristyl alcohol, glyceryl mono-stearate and sodium lauryl sulfate.

Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, 0.075 to 20% w/w. When formulated in an ointment, the active ingredients may be employed with either an oil-based, paraffinic or a water-miscible ointment base.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

Sterile injectable forms of the compositions described herein (e.g. for parenteral administration) may be aqueous or oleaginous suspension. These suspensions may be formulated according to techniques known in the art using suitable dispersing or wetting agents and suspending agents (including those described in the preceding paragraph). The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as a vegetable oil, for example arachis oil, olive oil, sesame oil or coconut oil, especially in their polyoxyethylated versions, or in mineral oil such as liquid paraffin. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an antioxidant such as butylated hydroxyanisol or alpha-tocopherol.

In another aspect, a compound of Formula I or a pharmaceutically acceptable salt thereof may be formulated in a veterinary composition comprising a veterinary carrier. Veterinary carriers are materials useful for the purpose of administering the composition and may be solid, liquid or gaseous materials which are otherwise inert. In the veterinary art and are compatible with the active ingredient. These veterinary compositions may be administered parenterally, orally or by any other desired route.

### Therapeutic Methods

In another aspect, the invention also provides a method of treating a disease in a subject in need thereof, comprising administering, alone or in combination therapy, a therapeutically effective amount of a compound of Formula I or a pharmaceutically acceptable salt thereof to the subject; wherein the disease is one that benefits from sGC stimulation or from an increase in the concentration of NO or cGMP or both, or from the upregulation of the NO pathway. The invention also provides a method of treating a disease in a subject in need thereof, comprising administering, alone or in combination therapy, a pharmaceutical composition comprising a compound of Formula I or a pharmaceutically acceptable salt thereof to the subject or a dosage form comprising the pharmaceutical composition, wherein the disease is one that benefits from sGC stimulation or from an increase in the concentration of NO or cGMP or both, or from the upregulation of the NO pathway.

In some embodiments, the compounds here disclosed are sGC stimulators that may be useful in the prevention and/or treatment of diseases characterized by undesirable reduced bioavailability of and/or sensitivity to NO, such as those associated with conditions of oxidative stress or nitrosative stress.

Increased concentration of cGMP leads to vasodilation, inhibition of platelet aggregation and adhesion, anti-hypertensive effects, anti-remodeling effects, anti-apoptotic effects, anti-inflammatory, anti-fibrotic effects, metabolic effects and neuronal signal transmission effects. Thus, sGC stimulators may be used to treat and/or prevent a range of diseases.

Specific diseases or disorders that benefit from sGC stimulation or from an increase in the concentration of NO or cGMP or both, or from the upregulation of the NO pathway, which therefore may be treated and/or prevented by administering an sGC stimulator of the invention (e.g., a compound of Formula I or a pharmaceutically acceptable salt thereof), include but are not limited to: Abetalipoproteinemia, achalasia (e.g., esophageal achalasia), acute respiratory distress syndrome (ARDS), adhesive capsulitis, age-related learning and memory disturbances, age-related memory loss, alcoholism, alopecia or hair loss, altitude sickness, Alzheimer's disease (including pre-Alzheimer's disease, mild to moderate Alzheimer's disease and moderate to severe Alzheimer's disease), amyotrophic lateral sclerosis (ALS or Lou Gehrig's disease), anal fissures, aneurysm, angina (e.g., stable or unstable angina pectoris, variant angina, Prinzmetal's angina, microvascular angina), anxiety or anxiety disorders, arginosuccinic aciduria, arterial and venous thromboses, arthritis, Asperger's syndrome, asthma and asthmatic diseases, ataxia, telangliectasia, atherosclerosis (e.g., atherosclerosis associated with endothelial injury, platelet and monocyte adhesion and aggregation, smooth muscle proliferation or migration), atrophic vaginitis, attention deficit disorder (ADD) and attention deficit hyperactivity disorder (ADHD), autism and disorders in the autism spectrum, benign prostatic hyperplasia (BPH) or hypertrophy or enlargement, bipolar disorder, bladder outlet obstruction, bladder pain syndrome (BPS), blepharitis, bone and carbohydrate metabolism disturbances, bone healing (e.g. bone healing following osteoclastic bone remodeling, osteoclastic bone resorption, new bone formation), brain aneurism, brain hypoxia, cancer metastasis, cerebral amyloid angiopathy (CAA) or congophilic angiopathy, cerebral autosomal-dominant arteriopathy with subcortical infarcts and leukoencephalopathy (CADASIL or CADASIL syndrome), cerebral perfusion, cerebral small vessel disease, cerebral vasospasm, chemo-brain, childhood disintegrative disorder, chronic bronchitis, chronic fatigue, chronic traumatic encephalopathy (CTE), ciliopathies, cirrhosis (e.g., liver cirrhosis, liver cirrhosis associated with chronic liver disease, primary biliary cirrhosis), CNS-disease related sexual dysfunction, CNS-disease related sleep disturbances, cognitive defect associated with Huntington's Disease, cognitive dysfunction, cognitive impairment (e.g., vascular cognitive impairment, mild cognitive impairment, cognitive impairment associated with diabetes, cognitive impairment associated with Multiple Sclerosis, cognitive impairment associated with obstructive sleep apnea, cognitive impairment associated with schizophrenia (CIAS), cognitive impairment associated with sickle cell disease, concussion, congenital myasthenic syndrome, connective tissue disease, consequences of cerebral infarction (apoplexia cerebri), conservation of blood substituents in trauma patients, CREST syndrome, Crohn's disease, cystic fibrosis (CF), delusional disorder, dementia (e.g., vascular dementia, post-stroke dementia, Lewy body dementia, dementia with frontal lobe degeneration, dementia with frontotemporal lobar degeneration, dementia with corticobasal degeneration, Creutzfeldt-Jakob dementia, HIV-dementia, multi-infarct dementia, post-operative dementia, strategic single-infarct dementia, HIV-associated dementia (including asymptomatic neurocognitive impairment (ANI), minor neurocognitive disorder (MND), HIV-associated dementia (HAD, also called AIDS dementia complex [ADC] or HIV encephalopathy), pre-senile dementia (mild cognitive impairment, MCI), mixed dementia, Binswanger's dementia (subcortical arteriosclerotic encephalopathy), Parkinson's Dementia), demyelination, depression, depressive disorder, dermatomyositis, diabetic angiopathy, diabetic macular edema, diabetic microangiopathies, diabetic ulcers or wounds (e.g., diabetic food ulcer), diseases associated with or related to metabolic syndrome (e.g. obesity, diabetes, insulin resistance, elevated fasting glucose, elevated fasting insulin, elevated lipids), diseases involving downregulated neurotransmitters, diseases involving impaired cerebral blood flow, diseases involving impaired neurodegeneration, diseases involving impaired synaptic function, diseases involving neuroinflammation, diseases involving neurotoxicity, diseases of the organs of the male and female urogenital system (benign and malignant), disturbances of concentration in children with learning and memory problems, Down syndrome, drug addiction, drug-induced psychosis, dry eye syndrome, Duchenne muscular dystrophy, Dupuytren's contracture, dyskinesia (e.g., acute dyskinesia, chronic or tardive dyskinesia, non-motor dyskinesia, levo-dopa induced dyskinesia (LID)), dysmenhorrea (e.g., primary dysmenhorrea, secondary dysmenhorrea), dyspaneuria, dysphagia, dystonia (e.g., generalized dystonia, focal dystonia, segmental dystonia, sexual dystonia, intermediate dystonia, acute dystonic reaction, genetic or primary dystonia), edema, elecrolyte disturbances (e.g., herkalemia, hyponatremia), emphysema, endometriosis, endothelial dysfunction or injury and diseases associated with endothelial dysfunction, erectile dysfunction, esophageal achalasia, Fabry Disease, female sexual dysfunction (e.g., female sexual arousal dysfunction), fibromyalgia, fibrosis (e.g., endomyocardial fibrosis, atrial fibrosis, cardiac interstitial fibrosis, cardiac fibrosis, pulmonary fibrosis, eye fibrosis, skin fibrosis, intestinal fibrosis, renal or kidney fibrosis, interstitial renal fibrosis, pulmonary fibrosis, idiopathic pulmonary fibrosis, progressive massive fibrosis of the lungs, liver fibrosis, mediastinal fibrosis, retroperitoneal fibrosis, arthrofibrosis, bone marrow fibrosis, myelofibrosis, osteomyelofibrosis, radiation-induced fibrosis, pancreatic fibrosis), Fragile X, functional dyspepsia, gastroparesis, Gaucher Disease, general disturbances of concentration, general psychosis, glaucoma, glioblastoma, glomerulopathies (e.g., glomerulonephritis, acute glomerulonephritis, glomerulosclerosis, focal segmental glomerulosclerosis), granulomas, head injury, hearing impairment (e.g., partial hearing loss, total hearing loss, partial deafness, total deafness, noise-induced hearing loss), heart disease (e.g., left ventricular myocardial remodeling, left ventricular systolic dysfunction, ischemic cardiomyopathy, dilatated cardiomyopathy, alcoholic cardiomyopathy, storage cardiomyopathies, congenital heart deffects, decreased coronary blood flow, diastolic or systolic dysfunction, coronary insufficiency, acute coronary syndrome, coronary artery disease, arrhythmias, reduction of ventricular preload, cardiac hypertrophy, right heart hypertrophy, disturbances of atrial and ventricular rhythm and heart conduction disturbances, atrioventricular blocks of degree I-III (AVB I-III), supraventricular tachyarrhythmia, premature ventricular contraction, atrial fibrillation, atrial flutter, ventricular fibrillation, ventricular flutter, ventricular tachyarrhythmia, torsade-de-pointes tachycardia, atrial and ventricular extrasystoles, AV-junction extrasystoles, sick-sinus syndrome, AV-node reentry tachycardia, Wolff-Parkinson-White syndrome, myocardial insufficiency, chronic, acute or viral myocarditis, cardiogenic shock, cardiac remodeling), heart failure (HF; e.g.: Heart failure with preserved ejection fraction (HFPEF), Heart failure with reduced ejection fraction (HFREF), acute heart failure, chronic heart failure, acute phases of an existing chronic heart failure (worsening HF), transient heart failure, post-acute heart failure, systolic heart failure, diastolic heart failure, congestive heart failure, acute decompensated heart failure, right ventricular failure, total heart failure, high output heart failure, heart failure with valvular defects, diabetic heart failure, heart failure/cardiorenal syndrome, right heart failure), high concentration of plasminogen activator inhibitor 1 (PA-1), high levels of fibrinogen and low density DLD, histiocytosis X, Huntington's disease or chorea (HD), hyperammonemia and related, hypertension (e.g., arterial hypertension, resistant hypertension, diabetic hypertension, idiopathic hypertension, essential hypertension, secondary hypertension, gestational hypertension, portal hypertension, systemic hypertension, pre-eclampsia, increased acute and chronic coronary blood pressure), hypertonia, hypertrophic scars, hypoactive sexual arousal disorder, hypoperfusion, impotence, Inflammatory bowel disease (e.g., Crohn's disease, Ulcerative Colitis), inflammation caused by cerebral malaria, inflammation caused by infectious disease, inflammatory response in perioprative care, platelet aggregation, intellectual disability, intermittent claudication, interstitial cystitis (IC), intradialytic hypotension, ischemia (e.g., cerebral ischemia, myocardial ischemia, thromboembolic ischemia, critical limb ischemia), keloids, kidney disease (e.g., chronic kidney disease, acute and chronic renal failure, acute and chronic renal insufficiency, sequelae of renal insufficiency, renal-insufficiency related to pulmonary enema, renal-insufficiency related to HF, renal-insufficiency related to uremia or anemia, primary kidney disease, congenital kidney disease, polycystic kidney disease progression, kidney transplant rejection, immune complex-induced kidney disease, abnormally reduced creatinine and/or water excretion, abnormally increased blood concentrations of urea, nitrogen, potassium and/or creatinine, altered activity of renal enzymes (e.g. glutamyl synthetase), altered urine osmolarity or urine volume, increased microalbuminuria, macroalbuminuria, lesions of glomeruli and arterioles, tubular dilatation, hyperphosphatemia, vascular kidney disease, renal cysts, renal edema due to HF), Korsakoff psychosis, leukocyte activation, levo-dopa induced addictive behavior, lichen sclerosus, lipid related disorders (e.g., excessive adiposity, excessive subcutaneous fat, hyperlipidemias, dyslipidemia, hypercholesterolemias, decreased high-density lipoprotein cholesterol (HDL-cholesterol), moderately elevated low-density lipoprotein cholesterol (LDL-cholesterol) levels, hypertriglyceridemias, hyperglyceridemia, hypolipoproteinanemias, sitosterolemia, fatty liver disease, liver steatosis or abnormal lipid accumulation in the liver, steatosis of the heart, kidney or muscle, sitosterolemia, xanthomatosis, Tangier disease), liver diseases (e.g., vascular liver disease, hepatic stellate cell activation, hepatic fibrous collagen and total collagen accumulation, liver disease of necro-inflammatory and/or of immunological, cholestatic liver disease associated with granulomatous liver diseases, cholestatic liver disease associated with liver malignancies, cholestatic liver disease associated with intrahepatic cholestasis of pregnancy, cholestatic liver disease associated with hepatitis, cholestatic liver disease associated with sepsis, cholestatic liver disease associated with drugs or toxins, cholestatic liver disease associated with graft-versus-host disease, cholestatic liver disease associated with post-liver transplantation, cholestatic liver disease associated with choledocholithiasis, cholestatic liver disease associated with bile duct tumors, cholestatic liver disease associated with pancreatic carcinoma, cholestatic liver disease associated with Mirizzi's syndrome, cholestatic liver disease associated with AIDS, cholangiopathy, cholestatic liver disease associated with parasites, cholestatic liver disease associated with schistosomiasis, hepatitis, non-alcoholic steatohepatitis (NASH), non-alcoholic fatty liver disease (NAFLD), hepatic vaso-occlusive disease (VOD), hepatic sinusoidal obstruction syndrome (SOS), hepatic encephalopathy), localized thrombosis, lower urinary tract syndromes (LUTS), lumbar spinal canal stenosis, lupus nephritis, lupus or Systemic Lupus Erythematosus, microalbuminuria, microcirculation abnormalities, migraines, minor neurocognitive disorder (MND), morphea, moyamoya, multiple lacunar infarction, multiple organ dysfunction syndrome (MODS), multiple organ failure (MOF), multiple sclerosis (MS, including clinically isolated syndrome (CIS), relapsing-remitting MS (RRMS), primary progressive MS (PPMS), secondary progressive MS (SPMS)), multiple system atrophy (MSA), myocardial infarction or heart attack (e.g., ST-segment elevation myocardial infarction, Non-ST-segment elevation myocardial infarction, old myocardial infarction), myopic choroidal neovascularization, naevi, narcotic dependence, nephropathies (e.g., diabetic nephropathy, non-diabetic nephropathy, nephritis, nephropathy induced by toxins, contrast medium induced nephropathy, diabetic or non-diabetic nephrosclerosis, nephrotic syndrome, pyelonephritis, nephrogenic fibrosis), neurodegenerative diseases, neurogenic bladder and incontinence, neuroinflammation, neurologic disorders associated with decreased nitric oxide production, neuromuscular diseases (e.g., Duchenne muscular dystrophy (DMD), Becker muscular dystrophy (BMD), limb girdle muscular dystrophies, distal myopathies, type I and type II myotonic dystrophies, facio-scapulo-peroneal muscular dystrophy, autosomal and X-linked Emery-Dreifuss muscular dystrophy, oculopharyngeal muscular dystrophy, amyotrophic lateral sclerosis, spinal muscle atrophy (SMA)), neuromyelitis optica, neuropathies (e.g., peripheral neuropathy, autonomic neuropathy, central nervous system neuropathy, chemotherapy induced neuropathy, diabetic neuropathy, painful neuropathies, neuropathic pain, non-painful neuropathies, painful diabetic neuropathy, non-painful diabetic neuropathy, neuropathies associated to a CNS disease (e.g., Multiple sclerosis, MS), radiation-induced neuropathy), neuropathic pain associated with shingles, neuropathic pain associated with spine surgery), obsessive compulsive disorder (OCD), obstructive thromboanginitis, obstructive uropathy, oesinophilic fasciitis, osteoporosis, overactive bladder, pain (e.g., acute pain, central pain syndrome, inflammatory pain, post-operative pain, tonic pain, visceral pain, claudication pain, orphan pain indications (e.g., Acetazolamide-responsive myotonia, Autoerythrocyte sensitization syndrome, Autosomal dominant Charcot-Marie-Tooth disease type 2V, Autosomal dominant intermediate Charcot-Marie-Tooth disease with neuropathic pain, Autosomal recessive limb-girdle muscular dystrophy type 2A, Channelopathy-associated congenital insensitivity to pain, Chronic pain requiring intraspinal analgesia, Complex regional pain syndrome, Complex regional pain syndrome type 1, Complex regional pain syndrome type 2, Congenital insensitivity to pain with hyperhidrosis, Congenital insensitivity to pain with severe intellectual disability, Congenital insensitivity to pain-hypohidrosis syndrome, Diffuse palmoplantar keratoderma with painful fissures, Familial episodic pain syndrome, Familial episodic pain syndrome with predominantly lower limb involvement, Familial episodic pain syndrome with predominantly upper body involvement, Hereditary painful callosities, Hereditary sensory and autonomic neuropathy type 4, Hereditary sensory and autonomic neuropathy type 5, Hereditary sensory and autonomic neuropathy type 7, Interstitial cystitis, Painful orbital and systemic neurofibromas-marfanoid habitus syndrome, Paroxysmal extreme pain disorder, Persistent idiopathic facial pain, Qualitative or quantitative defects of calpain, Tolosa-Hunt syndrome, pancreatitis, panic disorder, Parkinson's disease, Parkinsonism Plus, Parkinson's Dysphagia, pathological eating disorders, pelvic pain, peripheral vascular disease (e.g., peripheral arterial disease, peripheral arterial occlusive disease, peripheral embolism, peripheral perfusion disturbances), peritonitis, pervasive development disorder, Peyronie's disease, Picks syndrome, polychondritis, polymyositis, post herpetic neuralgia, post-traumatic head injury, post-traumatic stress disorder (PTSD), premature ejaculation, progressive nuclear palsy, prostate hypertrophy, pulmonary disease (e.g., plexogenic pulmonary arteriopathy, bronchoconstriction or pulmonary bronchoconstriction, vascular disease of the lung, chronic obstructive pulmonary disease (COPD), pulmonary capillary hemangiomatosis, lymphangiomatosis and compressed pulmonary vessels (e.g., due to adenopathy, tumor or fibrosing mediastinitis), pulmonary vascular remodeling, pulmonary hypertonia), pulmonary hypertension (PH, e.g., pulmonary arterial hypertension (PAH), primary PH, secondary PH, sporatid PH, pre-capically PH, idiopathic PH, PH associated with left ventricular disease, PH associated with HIV, PH associated with SCD, PH associated with thromoboembolism (chronic thromboembolic PH or CTEPH), PH associated with sarcoidosis, PH associated with chronic obstructive pulmonary disease, PH associated with acute respiratory distress syndrome (ARDS), PH associated with acute lung injury, PH associated with alpha-1-antitrypsin deficiency (AATD), PH associated with pulmonary emphysema (e.g., smoking induced emphysema), PH associated with lung disease, PH associated with hypoxemia, PH associated with scleroderma, PH associated with cystic fibrosis (CF), PH associated with left ventricular dysfunction, PH associated with hypoxemia, PH (WHO groups I, II, III, IV and V), PH associated with mitral valve disease, PH associated with pericarditis, PH associated with constrictive pericarditis, PH associated with aortic stenosis, PH associated with dilated cardiomyopathy, PH associated with hyperthrophic cardiomyopathy, PH associated with restrictive cardiomyopathy, PH associated with mediastinal fibrosis, PH associated with pulmonary fibrosis, PH associated with anomalous pulmonary venous drainage, PH associated with pulmonary veno-occlusive disease, PH associated with pulmonary vasculitis, PH associated with collagen vascular disease, PH associated with congenital heart disease, PH associated with pulmonary venous hypertension, PH associated with interstitial lung disease, PH associated with sleep-disordered breathing, PH associated with chronic airflow obstruction, PH associated with obstructive sleep apnea, PH associated with central sleep apnea, PH associated with mixed sleep apnea, PH associated with alveolar hypoventilation disorders, PH associated with chronic exposure to high altitude, PH associated with neonatal lung disease, PH associated with alveolar-capillary dysplasia, PH associated with sickle cell disease, PH associated with other coagulation disorders, PH associated with chronic thromboembolism), radiculopathy, Raynaud's disease, Raynaud's syndrome (primary or secondary), refractory epilepsy, Renpennings's syndrome, reperfusion injury (e.g., ischemia-reperfusion damage, ischemia-reperfusion associated with organ transplant), restenosis (e.g., restenosis developed after thrombolysis therapies, after percutaneous transluminal angioplasties (PTAs), after transluminal coronary angioplasties (PTCAs), after heart transplant or after bypass operations), retinopathies (e.g., diabetic retinopathy, non-diabetic retinopathy, non-proliferative diabetic retinopathy, proliferative vitroretinopathy, peripheral retinal degeneration, retinal vein occlusion), Rhett's disorder, rheumatoid or rheumatic disease (e.g., arthritis, rheumatoid arthritis), sarcoidosis, sarcoids, schistosomiasis, schizoaffective disorder, schizophrenia, schizophrenia with dementia, scleroderma (e.g., localized scleroderma or circumscribed scleroderma, systemic scleroderma), sclerosis (e.g. renal sclerosis, progressive sclerosis, liver sclerosis, primary sclerosing cholanginitis, sclerosis of the gastro-intestinal tract, hippocampal sclerosis, focal sclerosis, primary lateral sclerosis, osteosclerosis, otosclerosis, atherosclerosis, tuberous sclerosis, systemic sclerosis), sepsis or septic shock or anaphylactic shock, Sickle Cell Anemia, Sickle Cell Disease, Sjogren's syndrome, sleep-wake disorders, Sneddon's syndrome, spasms (e.g., coronary spasms, vascular spasms, spasms of the peripheral arteries), spinal cord injury, spinal muscular atrophy, spinal subluxations, spinocerebellar ataxias, Steel-Richardson-Olszewski disease (progressive supranuclear palsy), stroke, subarachnoid hemorrhage, subcortical arteriosclerotic encephalopathy, syncopes, tauopathies, tension, thalamic degeneration, thromboembolic or thrombogenic disorders, transient ischemic attacks (TIAs), traumatic brain injury, tubulointerstitial diseases, ulcers, uterine fibroids, vaginal atrophy, valve defects (e.g., mitral valve stenosis, mitral valve regurgitation, insufficiency or incompetence, aortic valve stenosis, aortic valve insufficiency, tricuspic valve insufficiency, pulmonary valve stenosis, pulmonar valve insufficiency, combined valvular defects), vascular disease of the brain, vascular disorder resulting from cardiac and renal complications, vascular leakage or permeability, vasculitis (e.g., thrombotic vasculitis, occlusive thrombotic vasculitis, Kawasaki disease, arteritis, aortitis), vaso-occlusive crisis, venus graft failure, wet age-related macular degeneration and Williams syndrome.

The term “disease”, as used herein refers to any deviation from or interruption of the normal structure or function of any body part, organ, or system that is manifested by a characteristic set of symptoms and signs and whose etiology, pathology, and prognosis may be known or unknown. The term disease encompasses other related terms such as disorder and condition (or medical condition) as well as syndromes, which are defined as a combination of symptoms resulting from a single cause or so commonly occurring together as to constitute a distinct clinical picture. In some embodiments, the term disease refers to an sGC, cGMP and/or NO mediated medical or pathological disease.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In some embodiments, the subject is a non-human animal such as a farm animal (e.g., a horse, cow, pig or sheep), or a companion animal or pet (e.g., a dog, cat, mice, rats, hamsters, gerbils, guinea pig or rabbit). In some embodiments, the subject is a human.

The invention also provides a method for treating one of the above diseases in a subject, comprising administering a therapeutically effective amount of a compound of Formula I or a pharmaceutically acceptable salt thereof to the subject in need of the treatment. Alternatively, the invention provides the use of a compound of Formula I or a pharmaceutically acceptable salt thereof in the treatment of one of these diseases in a subject in need of the treatment. Also included in the invention is the use of a compound of Formula I or a pharmaceutically acceptable salt thereof for the manufacture of medicament for treating one of the above diseases in a subject in need of the treatment. The invention further provides a method of making or manufacturing a medicament useful for treating one of these diseases comprising using a compound of Formula I or a pharmaceutically acceptable salt thereof.

The term “biological sample”, as used herein, refers to an in vitro or ex vivo sample, and includes, without limitation, cell cultures or extracts thereof; biopsied material obtained from a mammal or extracts thereof; blood, saliva, urine, faeces, semen, tears, lymphatic fluid, ocular fluid, vitreous humor, cerebrospinal fluid (CSF), or other body fluids or extracts thereof.

“Treat”, “treating” or “treatment” with regard to a disorder, disease, condition, symptom or syndrome, refers to abrogating or improving the cause and/or the effects (i.e., the symptoms, physiological or physical manifestations) of the disorder, disease, condition or syndrome. As used herein, the terms “treat”, “treatment” and “treating” also refer to the delay or amelioration or prevention of the progression (i.e. the known or expected progression of the disease), severity and/or duration of the disease or delay or amelioration or prevention of the progression of one or more symptoms (i.e. “managing” without “curing” the condition), resulting from the administration of one or more therapies.

In other embodiments, the invention provides a method of stimulating sGC activity in a biological sample, comprising contacting said biological sample with a compound or composition of the invention. Use of a sGC stimulator in a biological sample is useful for a variety of purposes known to one of skill in the art. Examples of such purposes include, without limitation, biological assays and biological specimen storage.

### Combination Therapies

The compounds and pharmaceutical compositions described herein can be used alone or in combination therapy for the treatment of a disease mediated, regulated or influenced by sGC, cGMP and/or NO.

As used herein, the terms “in combination” (as in the phrase “in combination therapy”) or “co-administration” can be used interchangeably to refer to the use of more than one therapy (e.g., one or more therapeutic agents). The use of the terms does not restrict the order in which therapies (e.g., therapeutic agents) are administered to a subject.

The compounds and pharmaceutical compositions described herein can be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

When used in combination therapy with other agents, a “therapeutically effective amount” of the compounds and pharmaceutical compositions described herein and of the other agent or agents will depend on the type of drug used. Suitable dosages are known for approved agents and can be adjusted by the skilled artisan according to the condition of the subject, the type of condition(s) being treated, and the amount of a compound described herein being used. In cases where no amount is expressly noted, an effective amount should be assumed. In some embodiments, co-administration or combination therapy encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such co administration also encompasses use of each compound in a sequential manner in either order.

When co-administration involves the separate administration of a first amount of a compound of Formula I and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of Formula I or a pharmaceutically acceptable salt thereof and the second therapeutic agent can be administered in any order within about 24 hours of each other, within about 16 hours of each other, within about 8 hours of each other, within about 4 hours of each other, within about 1 hour of each other or within about 30 minutes of each other.

Examples of other therapeutic agents that may be combined with a compound of Formula I or a pharmaceutically acceptable salt thereof, either administered separately, or in the same pharmaceutical composition include, but are not limited to:

(1) Endothelium-derived releasing factor (EDRF) or NO gas.

(2) NO donors including, but not limited to: a nitrosothiol, a nitrite, a sydnonimine, a NONOate, a N-nitrosamine, a N-hydroxyl nitrosamine, a nitrosimine, nitrotyrosine, a diazetine dioxide, an oxatriazole 5-imine, an oxime, a hydroxylamine, a N-hydroxyguanidine, a hydroxyurea or a furoxan. Some examples of these types of compounds include: glyceryl trinitrate (also known as GTN, nitroglycerin, nitroglycerine, and trinitrogylcerin), the nitrate ester of glycerol; sodium nitroprusside (SNP), wherein a molecule of nitric oxide is coordinated to iron metal forming a square bipyramidal complex; 3-morpholinosydnonimine (SIN-1), a zwitterionic compound formed by combination of a morpholine and a sydnonimine; S-nitroso-N-acetylpenicillamine (SNAP), an N-acetylated amino acid derivative with a nitrosothiol functional group; diethylenetriamine/NO (DETA/NO), a compound of nitric oxide covalently linked to diethylenetriamine; an m-nitroxymethyl phenyl ester of acetyl salicylic acid. More specific examples of some of these classes of NO donors include: the classic nitrovasodilators, such as organic nitrate and nitrite esters, including nitroglycerin, amyl nitrite, isosorbide dinitrate, isosorbide 5-mononitrate, and nicorandil; isosorbide; 3-morpholinosydnonimine; linsidomine chlorohydrate (“SIN-1”); S-nitroso-N-acetylpenicillamine (“SNAP”); S-nitrosoglutathione (GSNO), sodium nitroprus side, S-nitrosoglutathione mono-ethyl-ester (GSNO-ester), 6-(2-hydroxy-1-methyl-nitrosohydrazino)-N-methyl-1-hexanamine or diethylamine NONOate.

(3) Other substances that enhance cGMP concentrations, including, but not limited toprotoporphyrin IX, arachidonic acid and phenyl hydrazine derivatives.

(4) Nitric Oxide Synthase substrates, including, but not limited to L-arginine, n-hydroxyguanidine based analogs, such as N[G]-hydroxy-L-arginine (NOHA), 1-(3, 4-dimethoxy-2-chlorobenzylideneamino)-3-hydroxyguanidine, and PR5 (1-(3, 4-dimethoxy-2-chlorobenzylideneamino)-3-hydroxyguanidine); L-arginine derivatives (such as homo-Arg, homo-NOHA, N-tert-butyloxy- and N-(3-methyl-2-butenyl)oxy-L-arginine, canavanine, epsilon guanidine-carpoic acid, agmatine, hydroxyl-agmatine, and L-tyrosyl-L-arginine); N-alkyl-N′-hydroxyguanidines (such as N-cyclopropyl-N′-hydroxyguanidine and N-butyl-N′-hydroxyguanidine), N-aryl-N′-hydroxyguanidines (such as N-phenyl-N′-hydroxyguanidine and its para-substituted derivatives which bear —F, —Cl, -methyl, —OH substituents, respectively); guanidine derivatives such as 3-(trifluoromethyl) propylguanidine.

(5) Compounds which enhance eNOS transcription.

(6) NO independent heme-independent sGC activators, including, but not limited to: BAY 58-2667 (described in patent publication DE19943635); HMR-1766 (ataciguat, described in patent publication WO2000002851); S 3448 (2-(4-chloro-phenylsulfonylamino)-4,5-dimethoxy-N-(4-(thiomorpholine-4-sulfonyl)-phenyl)-benzamide (described in patent publications DE19830430 and WO2000002851); and HMR-1069 (from Sanofi-Aventis).

(7) Heme-dependent, NO-independent sGC stimulators including, but not limited to:

YC-1 (see patent publications EP667345 and DE19744026); riociguat (BAY 63-2521, Adempas®, described in DE19834044); nelociguat (BAY 60-4552, described in WO 2003095451); vericiguat (BAY 1021189, described in U.S. Pat. No. 8,420,656); BAY 41-2272 (described in DE19834047 and DE19942809); BAY 41-8543 (described in DE19834044); etriciguat (described in WO 2003086407); CFM-1571 (described in patent publication WO2000027394); A-344905, its acrylamide analogue A-350619 and the aminopyrimidine analogue A-778935;

other sGC stimulators described in one of publications US20090209556, U.S. Pat. No. 8,455,638, US20110118282 (WO2009032249), US20100292192, US20110201621, U.S. Pat. Nos. 7,947,664, 8,053,455 (WO2009094242), US20100216764, U.S. Pat. No. 8,507,512, (WO2010099054) US20110218202 (WO2010065275), US20130012511 (WO2011119518), US20130072492 (WO2011149921), US20130210798 (WO2012058132) and Tetrahedron Letters (2003), 44(48): 8661-8663; and IW1973 (praliciguat), IW1701 (olinciguat) and IW-6463.

(8) Compounds that inhibit the degradation of cGMP and/or cAMP, including, but not limited to:
PDE1 inhibitors, PDE2 inhibitors, PDE-3 inhibitors such as, for example, amrinone, milrinone, enoximone, vesnarinone, pimobendan, and olprinone, PDE4 inhibitors, such as, for example, rolumilast, PDE5 inhibitors, such as, for example, sildenafil and related agents such as avanafil, lodenafil, mirodenafil, sildenafil citrate, tadalafil, vardenafil and udenafil; alprostadil; dipyridamole and PF-00489791; PDE6 inhibitors, PDE9 inhibitors, such as, for example, PF-04447943, PDE10 inhibitors such as, for example, PF-02545920 (PF-10), and PDE11 inhibitors.

(9) Anticoagulants, including but not limited to:

coumarines (Vitamin K antagonists) such as warfarin, cenocoumarol, phenprocoumon and phenindione;

heparin and derivatives such as low molecular weight heparin, fondaparinux and idraparinux;

direct thrombin inhibitors such as argatroban, lepirudin, bivalirudin, dabigatran and ximelagatran; and

tissue-plasminogen activators, used to dissolve clots and unblock arteries, such as alteplase.

(10) Antiplatelet drugs, including, but not limited to topidogrel, ticlopidine, dipyridamoleand aspirin.

(11) Supplemental oxygen therapy.

(12) Alpha-1-adrenoceptor antagonists, including, but not limited to prazosin, indoramin, urapidil, bunazosin, terazosin and doxazosin; atrial natriuretic peptide (ANP), ethanol, histamine-inducers, tetrahydrocannabinol (THC) and papaverine.

(13) Bronchodilators, including, but not limited to:

short acting β₂ agonists, such as albutamol or albuterol and terbutaline;

long acting β₂ agonists (LABAs) such as salmeterol and formoterol;

anticholinergics such as pratropium and tiotropium; and

theophylline, a bronchodilator and phosphodiesterase inhibitor.

(14) Corticosteroids, including, but not limited to beclomethasone, methylprednisolone, betamethasone, prednisone, prednisolone, triamcinolone, dexamethasone, fluticasone, flunisolide, hydrocortisone, and corticosteroid analogs such as budesonide.

(15) Dietary supplements, including but not limited to omega-3 oils; folic acid, niacin, zinc, copper, Korean red ginseng root, ginkgo, pine bark, Tribulus terrestris, arginine, Avena sativa, horny goat weed, maca root, muira puama, saw palmetto, and Swedish flower pollen; vitamin C, Vitamin E, Vitamin K2; testosterone supplements, testosterone transdermal patch; zoraxel, naltrexone, bremelanotide and melanotan II.

(16) PGD2 receptor antagonists.

(17) Immunosuppressants, including, but not limited to cyclosporine, tacrolimus, rapamycin and other FK-506 type immunosuppressants, mycophenolate, mycophenolate mofetil.

(18) Non-steroidal anti-asthmatics, including, but not limited to:

β₂-agonists like terbutaline, metaproterenol, fenoterol, isoetharine, albuterol, salmeterol, bitolterol and pirbuterol;

β₂-agonist-corticosteroid combinations such as salmeterol-fluticasone, formoterol-budesonide, theophylline, cromolyn, cromolyn sodium, nedocromil, atropine, ipratropium, ipratropium bromide; and

leukotriene biosynthesis inhibitors such as zileuton or veliflapon.

(19) Non-steroidal anti-inflammatory agents (NSAIDs), including, but not limited to: propionic acid derivatives like alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen;

acetic acid derivatives such as indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac;

fenamic acid derivatives such as flufenamic acid, meclofenamic acid, mefenamic acid, niflumic acid and tolfenamic acid;

biphenylcarboxylic acid derivatives such as diflunisal and flufenisal;

oxicams such as isoxicam, piroxicam, sudoxicam and tenoxican;

salicylates such as acetyl salicylic acid and sulfasalazine; and

pyrazolones such as apazone, bezpiperylon, feprazone, mofebutazone, oxyphenbutazone and phenylbutazone.

(20) Cyclooxygenase-2 (COX-2) inhibitors, included, but not limited to celecoxib, rofecoxib, valdecoxib, etoricoxib, parecoxib and lumiracoxib; opioid analgesics such as codeine, fentanyl, hydromorphone, levorphanol, meperidine, methadone, morphine, oxycodone, oxymorphone, propoxyphene, buprenorphine, butorphanol, dezocine, nalbuphine and pentazocine.

(21) Adrenergic neuron blockers, including, but not limited to guanethidine and guanadrel.

(22) Imidazoline I-1 receptor agonists, including, but not limited to rimenidine dihydrogen phosphate and moxonidine hydrochloride hydrate.

(23) Potassium channel activators, including, but not limited to pinacidil.

(24) Dopamine D1 agonists, including, but not limited to fenoldopam mesilate; other dopamine agonists such as ibopamine, dopexamine and docarpamine.

(25) 5-HT2 antagonists, including, but not limited to ketanserin.

(26) Vasopressin antagonists, including, but not limited to tolvaptan.

(27) Calcium channel sensitizers, including, but not limited to levosimendan or activators such as nicorandil.

(28) Adenylate cyclase activators, including, but not limited to colforsin dapropate hydrochloride.

(29) Positive inotropic agents, including, but not limited to digoxin and metildigoxin; metabolic cardiotonic agents such as ubidecarenone; brain natriuretic peptides such as nesiritide.

(30) Drugs used for the treatment of erectile dysfunction, including, but not limited to alprostadil, aviptadil, and phentolamine mesilate.

(31) Drugs used for the treatment of Alzheimer's disease and dementias, including but not limited to:

acetyl cholinesterase inhibitors such as galantamine, rivastigmine, donepezil and tacrine; and

NMDA receptor antagonists such as memantine; and

oxidoreductase inhibitors such as idebenone.

(32) Psychiatric medications, including, but not limited to:

ziprasidone, risperidone, olanzapine, valproate;

dopamine D4 receptor antagonists such as clozapine;

dopamine D2 receptor antagonists such as nemonapride;

mixed dopamine D1/D2 receptor antagonists such as zuclopenthixol;

GABA A receptor modulators such as carbamazepine;

sodium channel inhibitors such as lamotrigine;

monoamine oxidase inhibitors such as moclobemide and indeloxazine; and

primavanserin, and perospirone.

(33) Drugs used for the treatment of movement disorders or symptoms, including, but not limited to:

catechol-O-methyl transferase inhibitors such as entacapone;

monoamine oxidase B inhibitors such as selegiline;

dopamine receptor modulators such as levodopa;

dopamine D3 receptor agonists such as pramipexole;

decarboxylase inhibitors such as carbidopa;

other dopamine receptor agonists such as pergolide, ropinirole, cabergoline;

ritigonide, istradefylline, talipexole; zonisamide and safinamide; and

synaptic vesicular amine transporter inhibitors such as tetrabenazine.

(34) Drugs used for the treatment of mood or affective disorders or OCD such as the following types:

tricyclic antidepressants such as amitriptyline, desipramine, imipramine, amoxapine, nortriptyline, doxepin and clomipramine;

selective serotonin reuptake inhibitors (SSRIs) such as paroxetine, fluoxetine, sertraline, trazodone and citralopram;

atypical antidepressants such as agomelatine;

selective norepinephrine reuptake inhibitors (SNRIs) such as venlafaxine, reboxetine and atomoxetine; dopaminergic antidepressants such as bupropion and amineptine.

(35) Drugs for the enhancement of synaptic plasticity, including, but not limited to:

nicotinic receptor antagonists such as mecamylamine; and

mixed 5-HT, dopamine and norepinephrine receptor agonists such as lurasidone.

(36) Drugs used for the treatment of ADHD such as amphetamine; 5-HT receptor modulators such as vortioxetine and alpha-2 adrenoceptor agonists such as clonidine.

(37) Nitric oxide synthase cofactors, including, but not limited to tetrahydrobiopterin, dihydrobiopterin and sapropterin.

(38) Blood glucose lowering medications (also referred as glycemic control medications or antidiabetic medications) including, but not limited to:

biguanides such as metformin;

sulfonylureas such as glyburide, glybenclamide, glipizide, gliclazide, gliquidone, glimepiride, atorvastatin calcium combined with glimerpiride, meglinatide, tolbutamide, chlorpropamide, acetohexamide, and tolazimide;

alpha-glucosidase inhibitors such as acarbose, epalrestat, voglibose, and miglitol;

insulin secretagoges such as repaglinide, mitiglinide and nateglinide;

thiazolidinediones such as rosiglitazone, troglitazone, ciglitazone, pioglitazone, englitazone, lobeglitazone sulfate and balaglitazone;

DPP-4 inhibitors (or DPP-IV inhibitors) such as sitagliptin, vildagliptin, saxagliptin, alogliptin, linagliptin, alogliptin benzoate combined with metformin or metformin hydrochloride, anagliptin, teneligliptin, atorvastatin calcium and glimepiride, empagliflozin combined with linagliptin, gemigliptin, sitagliptin phosphate monohydrate combined with pioglitazone hydrochloride, sitagliptin combined with pioglitazone, sitagliptin combined with atorvastatin calcium, and (2S,4S)-1-[2-(1,1-dimethyl-3-oxo-3-pyrrolidin-1-yl-propylamino)acetyl]-4-fluoro-pyrrolidine-2-carbonitrile (DBPR-108);

GLP-1 receptor agonists or incretin mimetics such as exenatide, dulaglutide, liraglutide, semaglutide, lixisenatide, lixisenatide combined with insulin glargine, albiglutide and pegapamodutide (TT-401), LY3298176 (dual glucose-dependent insulinotropic polypeptide (GIP) and GLP-1 receptor agonist);

SGLT2 inhibitors (SGLT2is) such as empagliflozin, empaglifozin combined with linagliptin, empagliflozin combined with metformin, ipragliflozin, ipragliflozin L-proline, tofogliflozin, sergliflozin etabonate, remogliflozin etabonate, ertugliflozin, ertugliflozin combined with sitagliptin, ertugliflozin combined with metformin, sotagliflozin, canagliflozin, canagliflozin combined with metformin or metformin hydrochloride, dapagliflozin, dapagliflozin combined with metformin or metformin hydrochloride and luseoglifozin, dapagliflozin combined with saxagliptin;

SGLT1 inhibitors or combinations of SGLT1 and SGLT2 inhibitors such as sotagliflozin;

insulin therapy such one of the many types of insulin, like insulin glulisine, insulin degludec, insulin lispro, insulin aspart, insulin glargine, insulin detemir, insulin isophane, insulin mixtard (human insulin containing both fast-acting (soluble) and long-acting (isophane) insulin, insulin degludec combined with insulin aspart, insulin human (rDNA origin) inhalation powder, recombinant human insulin, hepatic-directed vesicle insulin, insulin tregopi (IN-105), insulin degludec combined with liraglutide, insulin peglispro (LY-2605541) and nodlin; and

tolimidone (a lyn kinase activator).

(39) Blood pressure lowering medications (also known as anti-hypertensive medications), including, but not limited to:

diuretics such as thiazide diuretics, chlorothiazide, chlorthalidone, hydrochlorothiazide, bendroflumethiazide, cyclopenthiazide, methyclothiazide, polythiazide, quinethazone, xipamide, metolazone, indapamide, cicletanine, furosemide, toresamide, amiloride, spironolactone, canrenoate potassium, eplerenone, triamterene, acetazolamid and carperitide;

beta blockers such as acebutolol, atenolol, metoprolol, and nebivolol;

angiotensin-converting enzyme (ACE) inhibitors such as sulfhydryl-containing agents (for example, captopril, zofenopril), dicarboxylate-containing agents (for example, enalapril, quinapril, ramipril, perindopril, lisinopropil, and benazepril), phosphonate-containing agents (for example fosinopril), naturally occurring ACE inhibitors (for example, casokinins, lactokinins, lactotripeptides Val-Pro-Pro and Ile-Pro-Pro), alacepril, delapril, cilazapril, imidapril, temocapril, moexipril, lisinopril, combinations of lisinopril with hydrochlorothiazide, trandolapril and spirapril;

angiotensin II receptor blockers (ARBs) such as candesartan, losartan, losartan potassium-hydrochlorothiazide, valsartan, candesartan cilexetil, eprosaran, irbesartan, telmisartan, olmesartan medoxomil (or olmesartan), azilsartan medoxomil, azilsartan, amlodipine besylate combined with irbesartan, azilsartan combined with amlodipine besilate, cilnidipine combined with valsartan, fimasartan, irbesartan combined with atorvastatin, irbesartan combined with trichlormethiazide, losartan potassium combined with hydrochlorothiazide and/or amlodipine besylate, pratosartan, atorvastatin calcium combined with losartan potassium, nifedipine and candesartan cilexetil, sacubitril combined with valsartan or LCZ-696, angiotensin AT2 antagonist and TAK-591 and olmesartan medoxomil;

endothelin Receptor antagonists (ERAs) such as atrasentan, bosentan, sitaxentan, ambrisentan, actelion-1 (macitentan), Cyclo(D-trp-D-asp-L-pro-D-val-L-leu) (B Q-123), sparsentan and tezosentan disodium;

mineralocorticoid receptor antagonists (MRAs) such as spironolactone, amiloride hydrochloride combined with spironolactone, apararenone or MT-3995, eplerenone, and finerenone (BAY-94-8862);

calcium channel blockers such as amlodipine, aranidipine, azelnidipine, barnidipine, benidipine, cilnidipine, clevidipine, diltiazem, efonidipine, felodipine, lacidipine, lercanidipine, manidipine, nicardipine, nifedipine, nilvadipine, nimodipine, nisoldipine, nitrendipine, pranidipine, isradipine, verapamil, gallopamil, diltiazem, mibefradil, bepridil, fluspirilene and fendiline;

renin inhibitors such as aliskiren;

alpha blockers such as doxazosin and prazosin;

alpha-beta blockers such as carvedilol and labetalol;

central-acting agents such as clonidine, guanfacine and methyldopa;

vasodilators such as nitroglycerine, hydralazine and minoxidil; and

aldosterone antagonists such as finerenone, spironolactone and eplerenone.

(40) Anti-hyperlipidemic medications, including but not limited to:
statins such as atorvastatin fluvastatin, lovastatin, pitavastatin, pravastatin, rosuvastatin, and simvastatin;
combinations of statins with another agent such as amlodipine/atorvastatin, aspirin/pravastatin, ezetimibe/simvastatin, niacin/simvastatin, lovastatin/niacin, simvastatin/sitagliptin and atorvastatin/ezetimibe;
fibrates or fibric acid derivatives. Examples include, but are not limited to, fenofibrate, gemfibrozil, bezafibrate, ciprofibrate, clinofibrate and clofibrate;
niacin (or nicotinic acid);
bile acid sequestrants such as cholestyramine, colesevelam, colestilan and colestipol;
ezetimibe, lomitapide, phytosterols or orlistat; and
PCSK9 inhibitors such as alirocumab and evolocumab;

(41) Neprilysin inhibitors (also known as endopeptidase inhibitors or NEP inhibitors or enkephalinase inhibitors), including, but not limited to sacubitril, or the combination of sacubitril with valsartan; neprilysin inhibitors in development TD-1439 or TD-0714.

(42) Renoprotective drugs, including, but are not limited to:

Bardoxolone;

ACE inhibitors such as captopril;

ARBs such as losartan or irbesartan;

SGLT2 inhibitors such as canagliflozin,

GLP1 receptor agonists;

MRAs such as finerenone;

ERAs such as atrasentan; and

apoptosis signal-regulating kinase 1 (ASK1) inhibitors such as selonsertib.

(43) Hydroxyurea (HU, hydroxycarbamide).

(44) Anti-sickling agents, including, but not limited to hydroxyurea, voxelotor or GBT-440.

(45) Anti-adhesion therapies, including, but not limited, to blocking antibodies to P-selectin, E-selectin, VLA-4, VCAM-1.

(46) Glutamine.

(47) Erythropoietin (EPO), also known as hematopoietin or hemopoietin, including all its forms such as exogenous erythropoietin, recombinant human erythropoietin (rhEPO) or other erythropoiesis-stimulating agents (ESA) two examples being epoetin alfa and epoetin beta.

(48) Antibiotics, including but not limited to:

penicilin and its derivatives, including, but not limited to penicillin, amoxicillin, ampicillin, azlocillin, cloxacillin, penicillin G, penicillin V, procaine penicillin or benzathine penicillin amongst others.

cephalosporins such as cephalexin, cefadroxil, cefaclor, cefuroxime and cefexime;

macrolides such as erythromycin, clarithromycin, azithromycin, and roxithromycin;

tetracycline and its derivatives such as demeclocycline, doxycycline, minocycline, oxytetracycline and tetracycline;

sulfonamides, including, but not limited to, mafenide, sulfacetamide, sulfadiazine, silver sulfadiazine, sulfadimethoxine, sulfamethizole, sulfamethoxazole, sulfasalazine, trimethoprim-sulfamethoxazole (Co-trimoxazole), and sulfisoxazole; and

quinolones, including, but not limited to ciprofloxacin, enoxacin, gatifloxacin, gemifloxacin, levofloxacin, lomefloxacin, moxifloxacin, ofloxacin, and nalidixic acid.

(49) FXR agonists, including but not limited to obeticholic acid, cenicriviroc, emricasan, GR-MD-02, selonsertib and elafibranor.

(50) Thyroid receptor-beta agonists, including, but not limited to MGL-3196.

(51) Acetyl-CoA carboxylase inhibitors, including but not limited to GS-0976.

### Packaging and Kits

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g. preventing or treating one or more of the diseases and disorders described herein). The kit may optionally comprise a second pharmaceutical composition comprising one or more additional agents described herein for co therapy use, a pharmaceutically acceptable carrier, vehicle or diluent. The pharmaceutical composition comprising the compound described herein and the second pharmaceutical composition contained in the kit may be optionally combined in the same pharmaceutical composition.

### EXAMPLES

Combinations of substituents and variables envisioned by this invention are only those that result in the formation of stable compounds. All referenced patent and non-patent publications are each incorporated herein by reference in its entirety.

### Example 1: Demethylation of (3-(3-(5-fluoro-4-methoxypyrimidin-2-yl)-1H-pyrazol-5-yl)isoxazole) to yield 5-fluoro-2-(5-(isoxazol-3-yl)-1H-pyrazol-3-yl)pyrimidin-4-ol

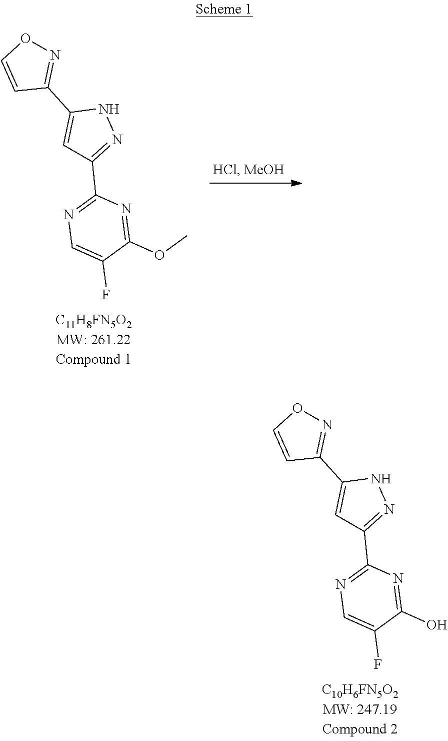 [SMILES: COc1nc(-c2cc(-c3ccon3)[nH]n2)ncc1F ; Oc1nc(-c2cc(-c3ccon3)[nH]n2)ncc1F]

The synthesis of Compound 1 (3-(3-(5-fluoro-4-methoxypyrimidin-2-yl)-1H-pyrazol-5-yl)isoxazole) has been previously described in International Patent Application Publication No. WO 2016/044447. In this demethylation process depicted in Scheme 1, Compound 1 (100 g, 0.383 mol, 1.0 equiv), methanol (1.2 L) and concentrated hydrochloric acid (189 mL, 37 wt. %, 2.30 mol, 6.0 equiv) were charged to a 5 L jacketed reaction vessel equipped with a nitrogen inlet-outlet, thermocouple, condenser, and overhead stirrer. The mixture was heated to 63 to 65° C. and stirred at 63 to 65° C. for a minimum of 22 hours, and a slurry was obtained. The reaction was complete by HPLC (Compound 1: Compound 2 area/area (a/a) %=1.0). The slurry was cooled to 20 to 25° C. over 30 minutes and held at 20 to 25° C. for 1 hour. The resulting slurry was filtered, and the filter cake was washed with methanol (0.5 L). The wet cake was dried under high vacuum at 45 to 55° C. over 16 hours until constant weight to furnish Compound 2 (5-fluoro-2-(5-(isoxazol-3-yl)-1H-pyrazol-3-yl)pyrimidin-4-ol) as an off-white solid (92.0 g, 96% yield, 99% pure by HPLC). ¹H-NMR (500 MHz, DMSO-d₆) δ ppm 14.20-14.60 (m, 1H); 13.10-13.75 (m, 1H); 8.95-9.20 (m, 1H); 8.05-8.30 (m, 1H); 7.35-7.75 (m, 1H); 6.85-7.25 (m, 1H).

### Example 2: Chlorination of 5-fluoro-2-(5-(isoxazol-3-yl)-1H-pyrazol-3-yl)pyrimidin-4-ol to Provide 3-(3-(4-chloro-5-fluoropyrimidin-2-yl)-1H-pyrazol-5-yl)isoxazole

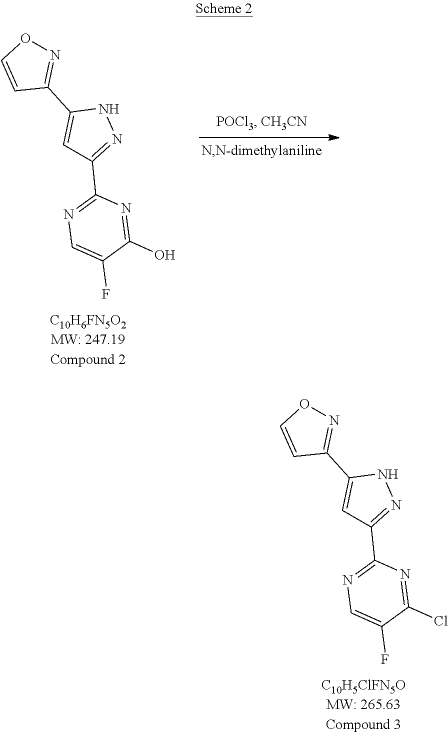 [SMILES: Oc1nc(-c2cc(-c3ccon3)[nH]n2)ncc1F ; Fc1cnc(-c2cc(-c3ccon3)[nH]n2)nc1Cl]

Compound 2 (72.0 g, 0.291 mol, 1.0 equiv), acetonitrile (0.72 L) and N,N-dimethylaniline (17.7 g, 0.146 mol, 0.5 equiv) were mixed in a 5 L jacketed reaction vessel with a nitrogen inlet, thermocouple, addition funnel, condenser, and overhead stirrer. The slurry was heated to 70 to 80° C. Phosphorous oxychloride (178 g, 1.16 mol, 4.0 equiv) was charged via an addition funnel over 1 hour, while maintaining the reaction temperature at 70 to 80° C. The mixture was stirred at 75 to 80° C. for a minimum of 8 hours. The reaction was complete by HPLC (Compound 2: Compound 3 a/a %=0.1). Then the mixture was cooled to 5 to 15° C. Water (0.40 L) was charged over 10 minutes via an addition funnel, while maintaining the reaction temperature below 15° C. The resulting slurry was stirred at 0 to 5° C. for 30 minutes and filtered. The filter cake was washed with a mixture of acetonitrile (0.36 L) and water (0.36 L), and then with water (0.36 L). The filter cake was dried under high vacuum at 45 to 55° C. over 16 hours until constant weight to furnish Compound 3 (3-(3-(4-chloro-5-fluoropyrimidin-2-yl)-1H-pyrazol-5-yl)isoxazole) as an off-white solid (75.3 g, 100% yield, 99% pure by HPLC). ¹H NMR (500 MHz, DMSO-d₆) δ ppm 14.36 (br s, 1H); 9.07 (br s, 1H); 9.02 (br s, 1H); 7.34 (br s, 1H); 7.01 (br s, 1H).

### Example 3: Coupling of (3-(3-(4-chloro-5-fluoropyrimidin-2-yl)-1H-pyrazol-5-yl)isoxazole) and 2-(aminomethyl)-1,1,1,3,3,3-hexafluoropropan-2-ol to Provide 1,1,1,3,3,3-hexafluoro-2-(((5-fluoro-2-(5-(isoxazol-3-yl)-1H-pyrazol-3-yl)pyrimidin-4-yl)amino)methyl)propan-2-ol

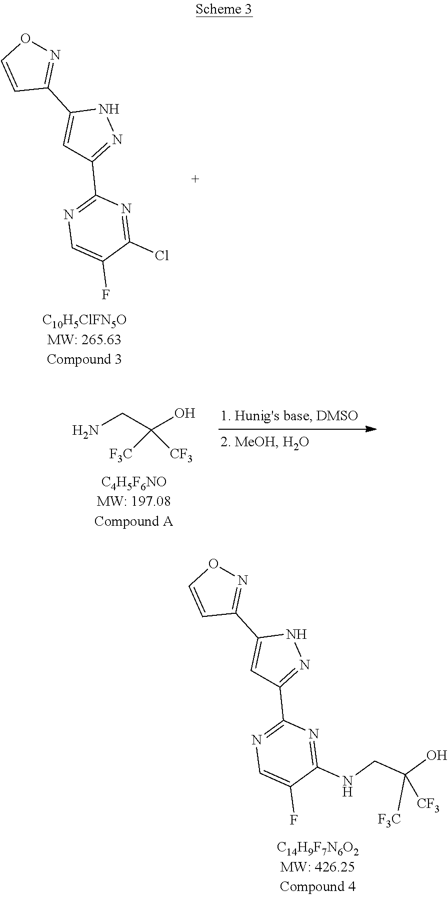 [SMILES: Fc1cnc(-c2cc(-c3ccon3)[nH]n2)nc1Cl ; NCC(O)(C(F)(F)F)C(F)(F)F ; OC(CNc1nc(-c2cc(-c3ccon3)[nH]n2)ncc1F)(C(F)(F)F)C(F)(F)F]

Compound 3 (3.0 g, 11.3 mol, 1.0 equiv), Compound A (8.9 g, 45.2 mol, 4.0 equiv) and dimethyl sulfoxide (12 mL) were charged to a 100 mL round bottom flask. Compound A (2-(aminomethyl)-1,1,1,3,3,3-hexafluoropropan-2-ol; CAS Registry No. 64139-78-2) is commercially available from at least AA Blocks LLC, Aldrich Partner Products, Azepine, Chemieliva Pharmaceutical and SynQuest, and therefore may be purchased. The reaction mixture was heated to 120 to 125° C. and held at 120 to 125° C. for 1 hour. The reaction was complete by HPLC (Compound 3: Compound 4 a/a %=0.3). The reaction mixture was cooled to 60 to 65° C. and water (60 mL) was added to the reaction mixture at 50 to 65° C. A thick slurry formed in a few minutes and the slurry was then cooled to 20 to 25° C. and stirred at 20 to 25° C. for 15 minutes. The slurry was filtered, and the filter cake was washed with water (150 mL). The filter cake was then dried under vacuum at 35 to 45° C. over 16 hours until constant weight to furnish crude Compound 4 (1,1,1,3,3,3-hexafluoro-2-(((5-fluoro-2-(5-(isoxazol-3-yl)-1H-pyrazol-3-yl)pyrimidin-4-yl)amino)methyl)propan-2-ol) as an off-white solid (4.7 g).

Crude Compound 4 (4.5 g) and methanol (45 mL) were charged to a 250 mL round bottom flask. The reaction mixture was heated to 58 to 63° C. until most solids dissolved. The solution was polish filtered to another 250 mL round bottom flask. The reaction mixture was then heated to 58 to 63° C. to obtain a solution, and water (30 mL) was charged while maintaining batch temperature above 60° C. The resulting slurry was cooled to 20 to 25° C. over 1 hour and held at 20 to 25° C. over 15 minutes. The slurry was filtered, and the filter cake was washed with a pre-mixed solution of methanol and water (15 mL/30 mL). The filter cake was then dried under vacuum at 35 to 45° C. over 16 hours to furnish Compound 4 as a white solid (4.2 g, 87% yield, 99% pure by HPLC). ¹H-NMR (500 MHz, Acetone-d₆) δ ppm 13.23 (br, s, 1H); 8.82 (s, 1H); 8.34 (d, J=2.90 Hz, 1H); 7.98 (br, s, 1H); 7.56 (br, s, 1H); 7.32 (s, 1H); 6.94 (s, 1H) 4.37 (s, 2H).

A procedure analogous to that described in Scheme 3 and in the preceding paragraphs could be used to introduce deuterium atoms in the alpha position of the amine chain by using a deuterated started amine. The preparation of the deuterated amine is described in Example 4.

### Example 4: Synthesis of deuterated 2-(aminomethyl)-1,1,1,3,3,3-hexafluoropropan-2-ol

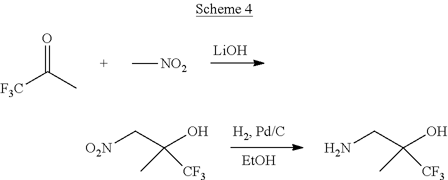

The synthesis of 3-amino-1,1,1-trifluoro-2-methylpropan-2-ol, as depicted in Scheme 4 above and as described in below, was previously reported in International Patent Application Publication No. WO 2013/038381.

### Step 1: Synthesis 1,1,1-trifluoro-2-methyl-3-nitropropan-2-ol

To LiOH (0.193 g, 8.06 mmol) in a 3-neck roundbottom flask was added water (25 nitromethane (3.76 ml, 81 mmol) and trifluoroacetone (7.95 ml, 89 mmol). Cetyltrimethylammonium chloride (3.8 g, 10.88 mmol) and MgSO₄ (1.9 g, 16.12 mmol) were then added and the resulting yellow solution stirred at 20-25° C. for 2 days. The reaction mixture was poured into diethyl ether (120 ml) and washed with water (3×200 ml) and brine (1×100 ml). The organic portion was dried over MgSO₄ and concentrated under reduced pressure to afford the title compound as a yellow liquid. ¹H NMR (CDCl₃, 400 MHz): δ 4.7 (1H d), δ 4.5 (1H, d), δ 3.7 (1H, broad), δ 1.6 (3H, 5).

### Step 2: Synthesis of 3-amino-1,1,1-trifluoro-2-methylpropan-2-ol hydrochloride

Pd/C was added (1 g) to a 200 ml glass vessel. Ethanol (50 ml, dry) was added cautiously under an atmosphere of CO₂. 1,1,1-Trifluoro-2-methyl-3-nitropropan-2-ol (10 g, 57.8 mmol) was dissolved in ethanol (50 ml, dry) and added to the glass vessel. The reaction mixture was put under a positive pressure of hydrogen (5 bar) at room temperature and hydrogenated for 2 days. The reaction mixture was filtered through Celite® (filter material) and washed with excess ethanol. The solvent was removed under reduced pressure to yield a colourless oil. The oil was dissolved in MeOH (50 ml) and treated dropwise with HCl (1 M) in MeOH (30 ml). The solution was left to stir for 30 minutes and concentrated under reduced pressure azeotroping with MeCN to afford the title compound as a waxy white solid. ¹H NMR (DMSO-d₆, 400 MHz) δ 8.3 (3H, broad s), 6.9 (1H, broad), 3.0 (2H, q), 1.4 (3H, s).

Reaction steps that are analogous to Steps 1 and 2 described above, as depicted in Scheme 5 below, can be used to prepare deuterated 2-(aminomethyl)-1,1,1,3,3,3-hexafluoropropan-2-ol (i.e., deuterated Compound A). Nitromethane-d₃ (CD₃NO₂) can be purchased from Cambridge Isotope Labs (Cat #DLM-30-25). Hexafluoroacetone can be purchased from Oakwood chemical (Cat #001507).

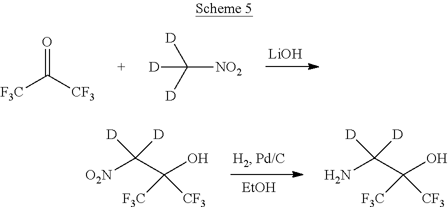

### Example 5: Alkylation of 1,1,1,3,3,3-hexafluoro-2-(((5-fluoro-2-(5-(isoxazol-3-yl)-1H-pyrazol-3-yl)pyrimidin-4-yl)amino)methyl)propan-2-ol with 2-fluorobenzyl bromide to Provide IW-1973

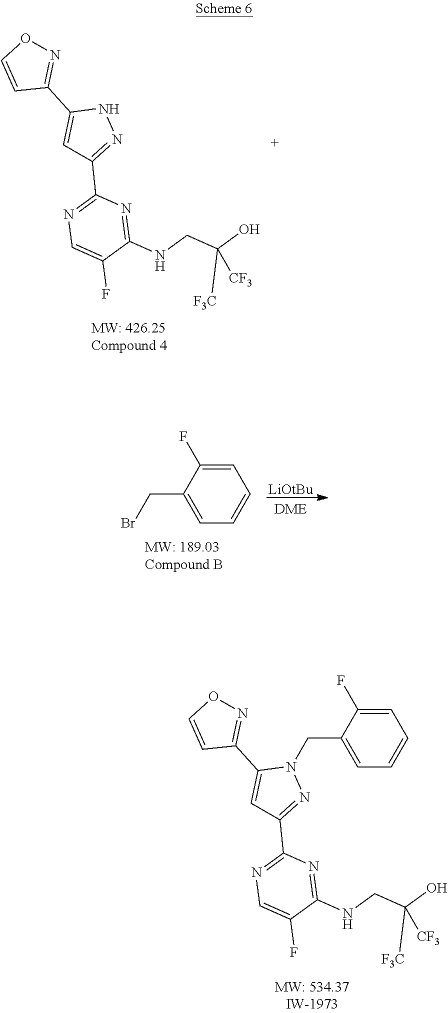 [SMILES: OC(CNc1nc(-c2cc(-c3ccon3)[nH]n2)ncc1F)(C(F)(F)F)C(F)(F)F ; Fc1ccccc1CBr ; OC(CNc1nc(-c2cc(-c3ccon3)n(Cc3ccccc3F)n2)ncc1F)(C(F)(F)F)C(F)(F)F ; "LiOtBu"]

To a solution of Compound 4 (88.0 mg, 0.206 mmol, 1.3 equiv) in anhydrous dimethoxyethane (1.5 mL) was added Compound B (2-fluorobenzyl bromide, 29.3 mg, 0.155 mmol, 1.0 equiv). Lithium tert-butoxide (23.1 mg, 0.289 mmol, 1.9 equiv) was added as a solid in one portion and heating continued to 65° C. (external temperature, internal temperature was not controlled.

After stirring for 16 hours (overnight) less than 10% of the 2-fluorobenzyl bromide remained based on HPLC analysis. The mixture was cooled to room temperature and ethyl acetate and water were added (10 mL each) and the layers separated. The aqueous layer was re-extracted with ethyl acetate (10 mL) and then the combined organic extracts washed with brine (10 mL), and concentrated to give crude material IW-1973. The crude material was purified by reverse phase chromatography on C18 column and the main fractions were pooled and concentrated under vacuum to give IW-1973 as a thick oil. The thick oil was dissolved in MeOH (1.5 mL) and water (1.5 mL) was added dropwise leading to precipitation of a fine white solid. The slurry was filtered and the wet cake was dried under vacuum at 40° C. overnight to give IW-1973 as a white solid (50.1 mg, 60% yield).

A procedure analogous to that depicted in Scheme 4 and the preceding paragraphs and the alternative procedures described below can be used to incorporate deuterium groups into the benzyl ring at different positions, depending on the starting 2-fluorobenzyl bromide used in each case (see below for some ways of making deuterated 2-fluorobenzyl bromides).

### Example 6: Synthesis of Various Deuterated 2-fluorobenzyl Bromides

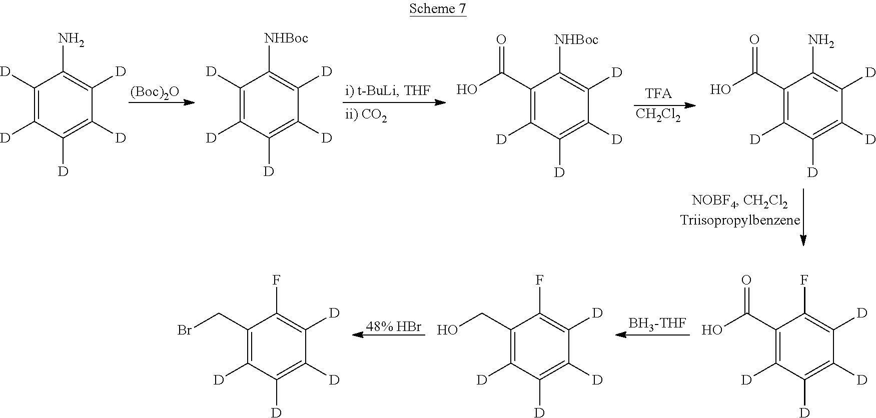

Various deuterated 2-fluorobenzyl bromide compounds can be prepared using the synthesis procedure depicted in Scheme 7 above. Aniline-ring-D5 can be purchased from Cambridge Isotope Labs (Cat #DLM-862-5). 2-Fluorobenzoic acid-ring-D5 can be purchased from CDN Isotopes (Cat #D-5991).

Following the same synthetic route, the following mono- and di-deuterated 2-fluorobenzyl bromides can be prepared using the synthetic procedure of Scheme 8 below. The requisite deuterated anilines can be synthesized following the methods described in Miura, Y. et al. J. Org. Chem. 1997, 62, 1188; Martins, A. and Lautens, M. Org. Lett. 2008, 10, 4351; and Liu, C. et al. Nature Communications 2018, 9, 1.

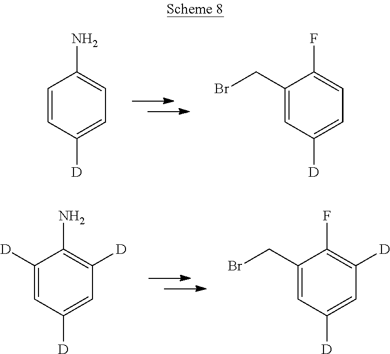

In addition, the following di-deuterated 2-fluorobenzyl bromide can be prepared using the synthetic procedure depicted in Scheme 9 below. The requisite deuterated aniline starting material can be purchased from CDN Isotopes (Cat #D-6903).

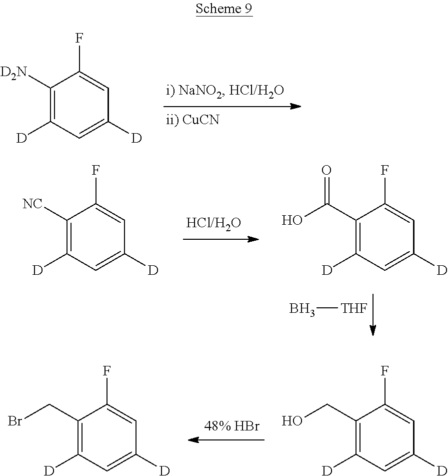

### Example 7: Synthesis of Compound 1-102

Compound 1-102 can be synthesized based on synthetic procedures depicted in Scheme 10, which is adapted and modified from synthesis procedures described in International Patent Application Publication No. WO 2011/044181.

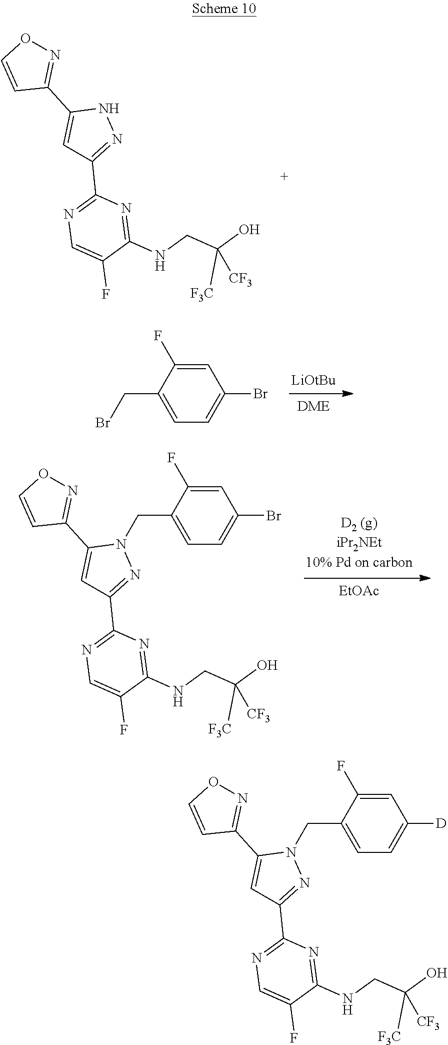

Without further description, it is believed that one of ordinary skill in the art can, using the preceding description and the illustrative examples, could make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound represented by Formula I: 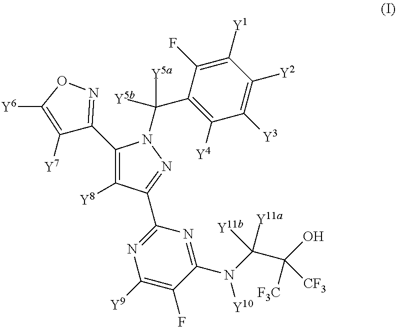
or a pharmaceutically acceptable salt thereof, wherein:
each of Y¹, Y², Y³, Y⁴, Y^{5a}, Y^{5b}, Y⁶, Y⁷, Y⁸, Y⁹, Y¹⁰, Y^{11a} and Y^{11b} is independently selected from hydrogen and deuterium; and
at least one of Y¹, Y², Y³, Y⁴, Y^{5a}, Y^{5b}, Y⁶, Y⁷, Y⁸, Y⁹, Y¹⁰, Y^{11a} and Y^{11b} is deuterium.

2. The compound of claim 1 or a pharmaceutically acceptable salt thereof, wherein at least one of Y¹, Y², Y³ and Y⁴ is deuterium.

3. The compound of claim 1 or a pharmaceutically acceptable salt thereof, wherein at least one of Y^{5a} and Y^{5b} is deuterium.

4. The compound of claim 1 or a pharmaceutically acceptable salt thereof, wherein at least one of Y⁶ and Y⁷ is deuterium.

5. The compound of claim 1 or a pharmaceutically acceptable salt thereof, wherein at least one of Y^{11a} and Y^{11b} is deuterium.

6. The compound of claim 1 or a pharmaceutically acceptable salt thereof, wherein each of Y¹, Y², Y³ and Y⁴ are the same.

7. The compound of claim 1 or a pharmaceutically acceptable salt thereof, wherein each of Y^{5a} and Y^{5b} are the same.

8. The compound of claim 1 or a pharmaceutically acceptable salt thereof, wherein each of Y⁶ and Y⁷ are the same.

9. The compound of claim 1 or a pharmaceutically acceptable salt thereof, wherein each of Y^{11a} and Y^{11b} are the same.

10. The compound of claim 1 or a pharmaceutically acceptable salt thereof, wherein Y¹ is deuterium.

11. The compound of claim 1 or a pharmaceutically acceptable salt thereof, wherein Y² is deuterium.

12. The compound of claim 1 or a pharmaceutically acceptable salt thereof, wherein Y³ is deuterium.

13. The compound of claim 1 or a pharmaceutically acceptable salt thereof, wherein Y⁴ is deuterium.

14. The compound of claim 1 or a pharmaceutically acceptable salt thereof, wherein Y^{11a} is deuterium.

15. The compound of claim 1 or a pharmaceutically acceptable salt thereof, wherein Y^{11b} is deuterium.

16. The compound of claim 1, wherein the compound is selected from any one of the compounds (Cmpd) set forth in the table below:

| Cmpd | Y¹ | Y² | Y³ | Y⁴ | Y^{11a} | Y^{11b} |
|---|---|---|---|---|---|---|
| I-101 | D | H | H | H | H | H |
| I-102 | H | D | H | H | H | H |
| I-103 | H | H | D | H | H | H |
| I-104 | H | H | H | D | H | H |
| I-105 | H | H | H | H | D | H |
| I-106 | H | H | H | H | H | D |
| I-107 | D | D | H | H | H | H |
| I-108 | D | H | D | H | H | H |
| I-109 | D | H | H | D | H | H |
| I-110 | D | H | H | H | D | H |
| I-111 | D | H | H | H | H | D |
| I-112 | H | D | D | H | H | H |
| I-113 | H | D | H | D | H | H |
| I-114 | H | D | H | H | D | H |
| I-115 | H | D | H | H | H | D |
| I-116 | H | H | D | D | H | H |
| I-117 | H | H | D | H | D | H |
| I-118 | H | H | D | H | H | D |
| I-119 | H | H | H | D | D | H |
| I-120 | H | H | H | D | H | D |
| I-121 | H | H | H | H | D | D |
| I-122 | D | D | D | H | H | H |
| I-123 | D | D | H | D | H | H |
| I-124 | D | D | H | H | D | H |
| I-125 | D | D | H | H | H | D |
| I-126 | D | H | D | D | H | H |
| I-127 | D | H | D | H | D | H |
| I-128 | D | H | D | H | H | D |
| I-129 | D | H | H | D | D | H |
| I-130 | D | H | H | D | H | D |
| I-131 | D | H | H | H | D | D |
| I-132 | H | D | D | D | H | H |
| I-133 | H | D | D | H | D | H |
| I-134 | H | D | D | H | H | D |
| I-135 | H | D | H | D | D | H |
| I-136 | H | D | H | D | H | D |
| I-137 | H | D | H | H | D | D |
| I-138 | H | H | D | D | D | H |
| I-139 | H | H | D | D | H | D |
| I-140 | H | H | D | H | D | D |
| I-141 | H | H | H | D | D | D |
| I-142 | H | H | D | D | D | D |
| I-143 | H | D | H | D | D | D |
| I-144 | H | D | D | H | D | D |
| I-145 | H | D | D | D | H | D |
| I-146 | D | H | D | D | D | H |
| I-147 | D | H | H | D | D | D |
| I-148 | D | H | D | H | D | D |
| I-149 | D | H | D | D | H | D |
| I-150 | D | H | D | D | D | H |
| I-151 | D | D | H | H | D | D |
| I-152 | D | D | H | D | H | D |
| I-153 | D | D | H | D | D | H |
| I-154 | D | D | D | H | H | D |
| I-155 | D | D | D | H | D | H |
| I-156 | D | D | D | D | H | H |
| I-157 | D | D | D | D | D | H |
| I-158 | D | D | D | D | H | D |
| I-159 | D | D | D | H | D | D |
| I-160 | D | D | H | D | D | D |
| I-161 | H | D | D | D | D | D |
| I-162 | D | H | D | D | D | D |
| I-163 | D | D | H | D | D | D |
| I-164 | D | D | D | D | D | D |

or a pharmaceutically acceptable salt thereof, wherein Y^{5a}, Y^{5b}, Y⁶, Y⁷, Y⁸, Y⁹, Y¹⁰, Y^{12a} and Y^{12b} are all H.

17. The compound of claim 1, wherein the compound is selected from any one of the following compounds or a pharmaceutically acceptable salt thereof:

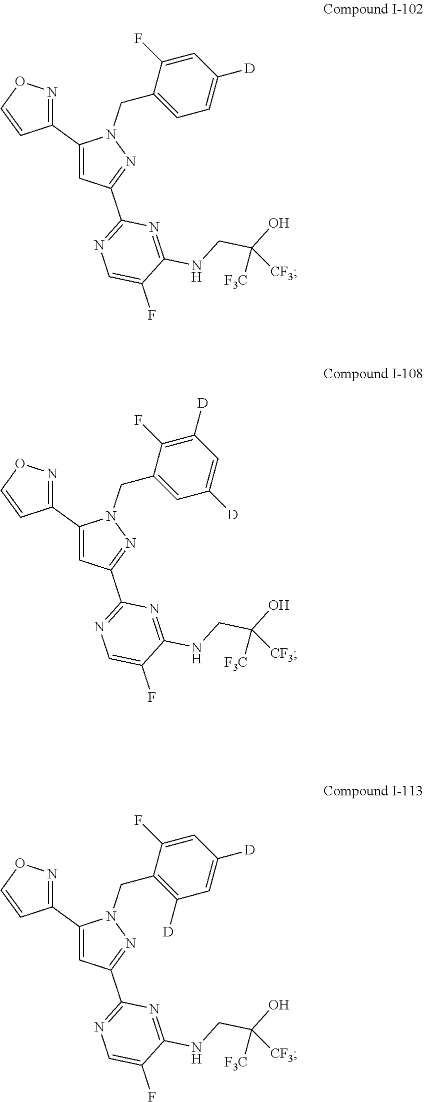 [SMILES: [2H]c1ccc(Cn2nc(-c3ncc(F)c(NCC(O)(C(F)(F)F)C(F)(F)F)n3)cc2-c2ccon2)c(F)c1 ; [2H]c1cc([2H])c(F)c(Cn2nc(-c3ncc(F)c(NCC(O)(C(F)(F)F)C(F)(F)F)n3)cc2-c2ccon2)c1 ; [2H]c1cc([2H])c(Cn2nc(-c3ncc(F)c(NCC(O)(C(F)(F)F)C(F)(F)F)n3)cc2-c2ccon2)c(F)c1]

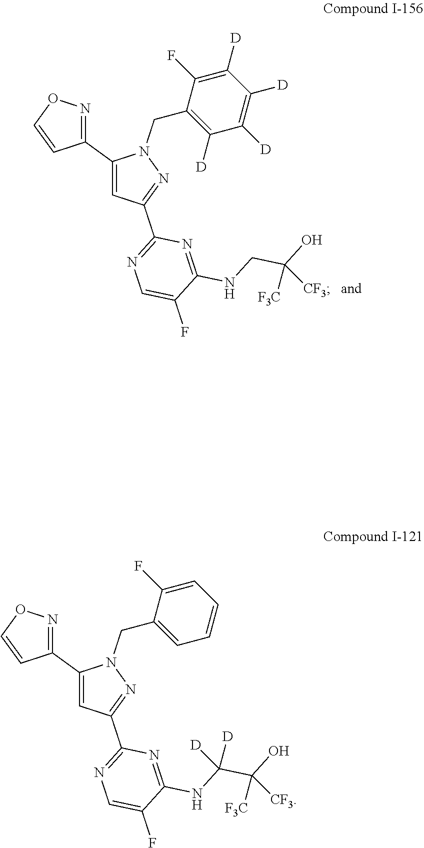 [SMILES: [2H]c1c([2H])c([2H])c(Cn2nc(-c3ncc(F)c(NCC(O)(C(F)(F)F)C(F)(F)F)n3)cc2-c2ccon2)c(F)c1[2H] ; "and" ; [2H]C([2H])(Nc1nc(-c2cc(-c3ccon3)n(Cc3ccccc3F)n2)ncc1F)C(O)(C(F)(F)F)C(F)(F)F]

18. The compound of claim 1, wherein
when any one of Y¹, Y², Y³ and Y⁴ is deuterium, the level of deuterium incorporation at each of Y¹, Y², Y³ and Y⁴ designated as deuterium is at least 52.5%, at least 75%, at least 82.5%, at least 90%, at least 95%, at least 97%, at least 98%, or at least 99%;
when any one of Y^{5a} and Y^{5b} is deuterium, the level of deuterium incorporation at each of Y^{5a} and Y^{5b} is at least 52.5%, at least 75%, at least 82.5%, at least 90%, at least 95%, at least 97%, at least 98%, or at least 99%;
when any one of Y⁶ and Y⁷ is deuterium, the level of deuterium incorporation at each of Y⁶ and Y⁷ is at least 52.5%, at least 75%, at least 82.5%, at least 90%, at least 95%, at least 97%, at least 98%, or at least 99%; or
when any one of Y^{11a} and Y^{11b} is deuterium, the level of deuterium incorporation at each of Y^{11a} and Y^{11b} is at least 52.5%, at least 75%, at least 82.5%, at least 90%, at least 95%, at least 97%, at least 98%, or at least 99%.

19-21. (canceled)

22. A pharmaceutical composition comprising a compound of claim 1 or a pharmaceutically acceptable salt thereof; and a pharmaceutically acceptable carrier.

23. A method of treating a disease or disorder in a subject in need thereof, comprising administering to the subject an effective amount of the compound of claim 1 or a pharmaceutically acceptable salt thereof; wherein the disease or disorder is a disease or disorder that benefits from sGC stimulation or from an increase in the concentration of NO or cGMP or both, or from the upregulation of the NO pathway; and wherein the disease or disorder is selected from hypertension, pulmonary hypertension (PH), pulmonary arterial hypertension (PAH), sickle cell disease, nonalcoholic steatohepatitis (NASH), gastrointestinal sphincter dysfunction, esophageal achalasia, a central nervous system (CNS) disease, an esophageal motility disorder, metabolic syndrome, heart failure with reduced ejection fraction (HFrEF), heart failure with preserved ejection fraction (HFpEF), and diabetic nephropathy.

24. (canceled)
